(19) United States
(12) Patent Application Publication
(10) Pub. No.: US 2023/0178211 A1
(43) Pub. Date: Jun. 8, 2023

(54) Method and System for Cardiac Pacing Therapy Guidance

(71) Applicants: Venster Medical, Inc., Menlo Park, CA (US); Peacs Investments B.V., (US)

(72) Inventors: Eelco Mattias van Dam, Nieuwerbrug aan den Rijn (NL); Peter Michael van Dam, Nieuwerbrug aan den Rijn (NL)

(21) Appl. No.: 17/923,888

(22) PCT Filed: May 6, 2021
(86) PCT No.: PCT/NL2021/050295

§ 371 Date: Nov. 7, 2022

(30) Foreign Application Priority Data
May 6, 2020 (NL) 2025520

(51) Int. Cl. G16H 20/40; A61N 1/365; A61N 1/02; G16H 50/50

(52) U.S. Cl. CPC G16H 20/40; A61N 1/365; A61N 1/025; G16H 50/50

(57) ABSTRACT

A method is provided for cardiac pacing therapy guidance including: receiving an 3D or anatomic model and/or processing thereof, receiving location information of at least one heart conduction feature and/or processing thereof, receiving location information of at least one cardiac vein and/or processing thereof, receiving ECG recording data and/or processing thereof, determining a heart activation map relative to the model, and determining potential implant areas for implantation of at least one pacing system lead, such as for the purpose of outputting such information relating to the model and the potential implant areas for displaying thereof.

### CROSS-REFERENCE TO RELATED APPLICATIONS

This application is the United States national phase of International Application No. PCT/NL2021/050295 filed May 6, 2021, and claims priority to The Netherlands Patent Application No. 2025520 filed May 6, 2020, the disclosures of each of which are hereby incorporated by reference in their entireties.

### BACKGROUND OF THE INVENTION

### Field of the Invention

The present invention relates to a method, such as implemented on a computer, providing cardiac pacing therapy guidance, such as during pacing system implant and/or implanted pacing system functioning optimization, for performing heart function optimization, such as comprising pacing system lead positioning guidance, stimulation timing guidance and/or stimulation sequence guidance. Furthermore, the present invention relates to a cardiac pacing therapy guidance system, such as comprising on a computer, providing cardiac pacing therapy guidance, such as during pacing system implant and/or implanted pacing system functioning optimization, for performing heart function optimization, such as comprising pacing system lead positioning guidance, stimulation timing guidance and/or stimulation sequence guidance.

### Description of Related Art

It is known to use pacemakers for regulating heart rhythms or heart rates. Pacemakers are capable of pacing up a heart when it beats to slowly or even slow it down when it beats to quickly. However, it is difficult to know where to attach electrical contacts of a pacemaker relative to the heart for best functioning, or even reliably indicate what patients actually benefit from a pacemaker.

### SUMMARY OF THE INVENTION

In order to provide better therapy, the present invention provides a method, such as implemented on a computer, providing cardiac pacing therapy guidance, such as providing input for pacing system implant and/or implanted pacing system functioning optimization, for performing heart function optimization, such as comprising pacing system lead positioning guidance, stimulation timing guidance and/or stimulation sequence guidance, the method comprising steps of:
receiving (100) an 3D or anatomic model and/or processing thereof, such as a torso model and/or a heart model,
receiving (400), such as by estimation or scanning data, location information of at least one heart conduction feature and/or processing thereof, such as the base of the papillary muscles, the right free wall moderator band connection, the right apical septal position, the mute apical left septal position and/or the basal left septal position,
receiving (500) location information of at least one cardiac vein and/or processing thereof, such as by estimation or scanning data,
receiving (600) ECG recording data and/or processing thereof, preferably with corresponding 3D torso information, of a currently performed ECG,
determining (700) a heart activation map relative to the model, such as comprising steps of updating of electrophysiological properties of the model,
determining (800) potential implant areas for implantation of at least one pacing system lead, such as for the purpose of outputting such information relating to the model and the potential implant areas for displaying thereof.

### BRIEF DESCRIPTION OF THE DRAWINGS

FIG. 1 is a flowchart indicative of a method according to preferred embodiments of the present invention;

FIG. 2 is a flowchart indicating details of step 100 according to a preferred embodiment of the present invention;

FIG. 3 is a flowchart relating to step 200 according to a preferred embodiment of the present invention;

FIG. 4 is a flowchart relating to step 300 according to a preferred embodiment of the present invention;

FIG. 5 shows a flowchart relating to step 400 according to a preferred embodiment of the present invention;

FIG. 6 shows a flowchart relating to step 500 according to a preferred embodiment of the present invention;

FIG. 7 shows a flowchart relating to the step 600 according to a preferred embodiment of the present invention;

FIG. 8 shows a flowchart relating to step 700 according to a preferred embodiment of the present invention;

FIG. 9 shows a flowchart relating to step 800 according to a preferred embodiment of the present invention;

FIG. 10 shows a flowchart relating to step 900 according to a preferred embodiment of the present invention;

FIG. 11 shows a flowchart relating to step 1000 according to a preferred embodiment of the present invention;

FIG. 12 shows a flowchart relating to step 1020 according to a preferred embodiment of the present invention;

FIG. 13 shows a flowchart relating to step 1030 according to a preferred embodiment of the present invention;

FIG. 14 shows a flowchart relating to step 1030 according to a preferred embodiment of the present invention;

FIG. 15 shows a flowchart relating to step 1030 according to a preferred embodiment of the present invention;

FIG. 16 is a flowchart according to an embodiment of the present invention;

FIG. 17 shows 3D positioning of the heart according to an embodiment of the present invention;

FIG. 18 is a model of a torso with a heart with blood cavities merged with an MRI image according to an embodiment of the present invention;

FIG. 19 is an x-ray of the heart showing veins therein according to an embodiment of the present invention;

FIG. 20 is a cross-section of the heart showing electrical activation sites of the His-Purkinje system according to an embodiment of the present invention;

FIG. 21 is a 3D heart torso model combination according to an embodiment of the present invention;

FIG. 22A is an schematic drawing of cardiac areas with scar according to step 730 according to an embodiment of the present invention;

FIG. 22B is a display of an estimated activation map according to an embodiment of the present invention;

FIG. 23 is an example of a preferred prediction parameter map according to an embodiment of the present invention;

FIGS. 24-27 are each 3D models of the heart showing different activation times according to an embodiment of the present invention;

FIG. 28 is a 3D model of the heart showing a localized lead position, according to an embodiment of the present invention;

FIG. 29 is an example of a prediction parameter map according to an embodiment of the present invention;

FIG. 30 is a graph of predicted synchrony at various lead locations according to an embodiment of the present invention;

FIG. 31 is a schematic drawing showing electrode locations for ECG according to an embodiment of the present invention;

FIG. 32 is a recorded ECG according to an embodiment of the present invention;

FIG. 33 is system for performing a computer implemented method according to the present invention according to an embodiment of the present invention;

FIG. 34 shows a flowchart relating to the steps comprised by step 750 according to a preferred embodiment of the present invention;

FIG. 35 is a 3D model of the heart showing regional sites of early or initial activation associated with the Purkinje activation according to an embodiment of the present invention;

FIG. 36 is a schematic drawing representative of regions of the heart according to an embodiment of the present invention; and

FIGS. 37-40 are representations of activation maps wherein a part of the map representing such region is related to a percentage of mass activated in that part representing such region according to an embodiment of the present invention.

### DETAILED DESCRIPTION

It is advantageous of the invention, aspects or embodiments thereof, that it can be rendered or shown, preferably based on the information as determined to create or used for the activation map, at what time or timing distinct parts of the atria walls to be activated for each heart beat are activated. This provides concrete information about differences in timing of the distinct parts of the atria walls that were not available in the prior art. The activation map is a set of data representing such parts of the atria walls including activation time or timing data of such respective parts of the atria walls. A rendered activation map shows the actual timings in the 3D rendering. Another representation of the activation map may include a graphs relating to predetermined wall segments and/or a relative mass of an activated part thereof at its activation time or timing. This very clearly provides insights in timing differences between such wall segments, such as RV free wall, RV septum, LV septum, LV anterior and/or LV posterior, just as such differences are visible in the 3D activation map.

A method according to the present invention provides advantages including the following. A major advantage is providing improved pacing therapy relating to synchrony of the heart function. During implantation or placement of a pacing system, such as a pacemaker, use of the present invention provides an improved or optimal lead position of the pacing system leads at or in the heart, such as in arteries, or cavities.

The present invention or embodiments thereof advantageously provide creation of the 3D or anatomic model, preferably comprising an electrophysiological model, further preferably based on patient specific anatomic data. With creation of the model placement of the leads is reached, preferably with an iterative approach. Achieved goals include an optimal lead positioning relative to the advance of me or heart, and optimal stimulation timing, and/or an optimal stimulation sequence. The model may be optimized based on additional new information, such as a new lead configuration and/or new ECG information. A further advantage is guidance relative to synchrony of the heart. To this end, synchrony may be calculated for respectively considered lead configurations and/or timings. Further preferably, a method according to the invention may be used, possibly while leaving out respective steps, to perform selection of a patient for cardiac pacing therapy to exclude patients of which such a method indicates such prospective patients to be non-beneficial with respect to the cardiac pacing therapy, even if such a patient has a QRS duration less than 150 ms. As such, the present invention provides the advantage of avoiding unnecessary cardiac pacing therapy and thus unnecessary implants.

Embodiments as described below further preferably achieve one or more of the following advantages. A result of the method may be a synchrony guidance or a determination of a target position at which to position a first or a further pacing system lead. Based thereon, the lead may be guided to the target position using further steps or reiterations thereof of the invention or embodiments. With a reiteration of a respective step, the synchrony guidance or prediction may be confirmed.

According to an embodiment, information of the determined model, calculations, results thereof, placed lead locations and applied pacing system settings are preferably stored in a data store for later use as a set of data indicated as pacing system, such as CRT, implantation data or model.

Further preferred embodiments as described below provides steps for improving the pacing system implantation data or model by modifying the pacing system settings after implantation thereof. To this end, which embodiment is based on loading of the pacing system implantation data or model and performing further steps by simulating and/or testing such settings in the model and/or in vivo for confirmation. Settings such embodiments are preferably envisioned to modify comprise stimulation timing, stimulation sequence that may be indicated by AV-timing, VV-timing and/or sequence of multiple sites stimulation.

According to a first preferred embodiment of the invention, the method comprises steps of determining a set of candidate positions for the at least one pacing system lead, preferably determining a target position for the pacing system lead from the set of candidate positions. With this, for preferably each potential cardiac pacing configuration, also definable as locations of the possible cardiac pacing leads and possible timings of the cardiac pacing leads, heart synchrony indices are determined. Envisioned ways thereof are described in greater detail below relating to the drawings.

According to a further preferred embodiment, the method comprises steps of processing (1000) scanning or recording data or signals pertaining to at least one pacing system lead activation during guidance thereof towards one of the set of candidate positions, preferably the target position, preferably including location information thereof relating to the location of the pacing system lead at the time of the activation.

With the applications of this embodiment, a feedback signal, such as by means of a representation in a graphical user interface may preferably provide feedback as to the position of the lead during the process of implanting the lead.

According to a further preferred embodiment, the steps of processing scanning or recording information comprise steps of processing (1020) vectorcardiogram, VCG, data or signals pertaining to the at least one activation of the pacing system lead. This provides the advantage that the lead may be located based on the QRS axes or the mean QRS axis, such as in a way as described in a co-pending application pct/n12017/050225 that is herein incorporated by reference.

According to a further preferred embodiment, the steps of processing scanning or recording information comprises steps of processing inverse ECG data or signals pertaining to the at least one activation of the pacing system lead. With this, preferably a simulated VCG is compared with a VCG from simulated isochrones, further preferably taking into account information relating to scar tissue, such as in a way as described in a co-pending application pct/n12016/050728 that is herein incorporated by reference.

According to a further preferred embodiment, the steps of processing scanning or recording information comprises steps of processing pacing spike data or signals, such as by localization, such as by localization of at least one pacing spike from the QRS of the ECG, pertaining to the at least one activation of the pacing system lead. Such a pacing spike is substantially the polar, which may be indicated as a little vector. An estimation of the location is preferably made using the model of the torso and heart as a volume conductor model, further preferably while applying a nonlinear estimation procedure.

According to a further preferred embodiment, the method comprises steps of including at least one intracavitary structure, such as papillary muscles or moderator band, preferably comprising information as to position, size and orientation of such structures. Preferably, scanning to obtain such information is performed by means of MRI or CT scanning methods and/or received from a preceding scan from a data store. In case such scanning results are not available, use can be made of an estimation model based on characteristics that may be available such as size of the torso, age, further physical characteristics of the patient and/or preferably a 3D recording of the torso. Based on such characteristics, a best fitting standardized model may be obtained from the data store. Further preferably, it is envisaged that measurements made with an echo device are used for assembling the model.

According to a further preferred embodiment, the method comprises steps of including (300) data pertaining to dysfunctional or traumatized myocardial tissue, such as areas of scar or ischemic tissue if present in the myocardial tissue. Preferably, scanning to obtain such information is performed by means of MRI or CT scanning methods and/or received from a preceding scan from a data store. In case such scanning results are not available, use can be made of an estimation model based on characteristics that may be available such as size of the torso, age, further physical characteristics of the patient and/or preferably a 3D recording of the torso. Based on such characteristics, a best fitting standardized model may be obtained from the data store. Further preferably, it is envisaged that measurements made with an echo device are used for assembling the model.

According to a further preferred embodiment, the method comprises steps of determining whether the at least one pacing lead is positioned at the target position or within a predetermined deviation threshold thereof.

Further preferably, according to the invention, an aspect provides a method, such as implemented on a computer, providing cardiac pacing therapy guidance, such as during pacing system functioning optimization, for performing heart function optimization, such as stimulation timing guidance and/or stimulation sequence guidance, the method comprising steps of:
receiving pacing system, such as CRT, implantation data from a data store and/or processing thereof,
receiving (600) ECG recording data and/or processing thereof, preferably with corresponding 3D torso information, of a currently performed ECG,
determine cardiac pacing therapy value set, such as comprising a synchrony value,
outputting pacing system settings to one of a set of test settings, preferably until all of the settings of the set of test settings have been performed, for the pacing system to be programmed to the respective setting,
displaying the determined cardiac pacing therapy value set on a display device.

With this, advantages as indicated in the above are achieved as well as the advantage that functioning of the pacing system may be improved after implantation thereof by altering its set of settings, such as stimulation timing and stimulation sequence, such as AV-timing, VV-timing and/or sequence of multiple sites stimulation.

According to a further preferred embodiment, the method comprises steps of outputting a setting identification identifying the test settings identified as best for applying the settings to the pacing system.

A further aspect according to the present invention relates to a cardiac pacing therapy guidance system, such as comprising on a computer, providing cardiac pacing therapy guidance, such as during pacing system implant and/or implanted pacing system functioning optimization, for performing heart function optimization, such as comprising pacing system lead positioning guidance, stimulation timing guidance and/or stimulation sequence guidance, the system comprising:
a processing unit,
a with the processing unit functionally coupled memory,
a 3D or anatomic model receiver, the model preferably being a torso model and/or a heart model,
a location information receiver of at least one heart conduction feature, such as the base of the papillary muscles, the right free wall moderator band connection, the right apical septal position, the mute apical left septal position and/or the basal left septal position,
a location information receiver for receiving (500) location information of at least one cardiac vein, such as by estimation or scanning data,
an ECG receiver for receiving (600) ECG recording data, preferably with corresponding 3D torso information, of a currently performed ECG,
a heart activation map determining module relative to the model, such as comprising steps of updating of electrophysiological properties of the model,
a potential implant areas determining module, for the purpose of determining the areas for implantation of at least one pacing system lead, such as for the purpose of outputting such information relating to the model and the potential implant areas for displaying thereof.

Such further aspect according to the present invention provides advantages as indicated in the above in relation to the method aspect.

According to a preferred embodiment, the system comprises a set of candidate positions determining module, preferably comprising a target position determining module. Such candidate positions determining module performs respective steps of the indicated method.

According to a preferred embodiment, the system comprises an ECG device for recording of respective ECG sessions, preferably a standardized ECG session, further preferably a standardized 12 lead ECG session. Such ECG device provides the respective ECG recording data to the ECG receiver.

According to a further preferred embodiment, the system comprises a 3D imaging module for recording of 3D torso information, of the torso to which a respective EGG is performed. Such 3D imaging module is equipped to record 3D imaging data of the torso of the patient subject to the cardiac pacing therapy. The 3D imaging module or the processing unit is preferably equipped with ECG lead recognition routines to determine the locations thereof relative to the torso, preferably subsequently relative to the 3D or anatomic model.

According to a further preferred embodiment, the system comprises an output means for outputting of a user interface, preferably a graphical user interface for displaying any information processed in or resulting from a method according to the invention.

Further preferably, the system comprises input means for inputting user operations, such as on the graphical user interface in order to operate the system in order to function with a method according to the invention. Such input means may comprise a mouse and/or keyboard, touchscreen or other per se known computer input peripherals.

Further advantages, features and details of the present invention will be further elucidated on the basis of a description of one or more preferred embodiments with reference to the accompanying figures. Similar yet not necessarily identical parts of different preferred embodiments may be indicated with the same reference numerals.

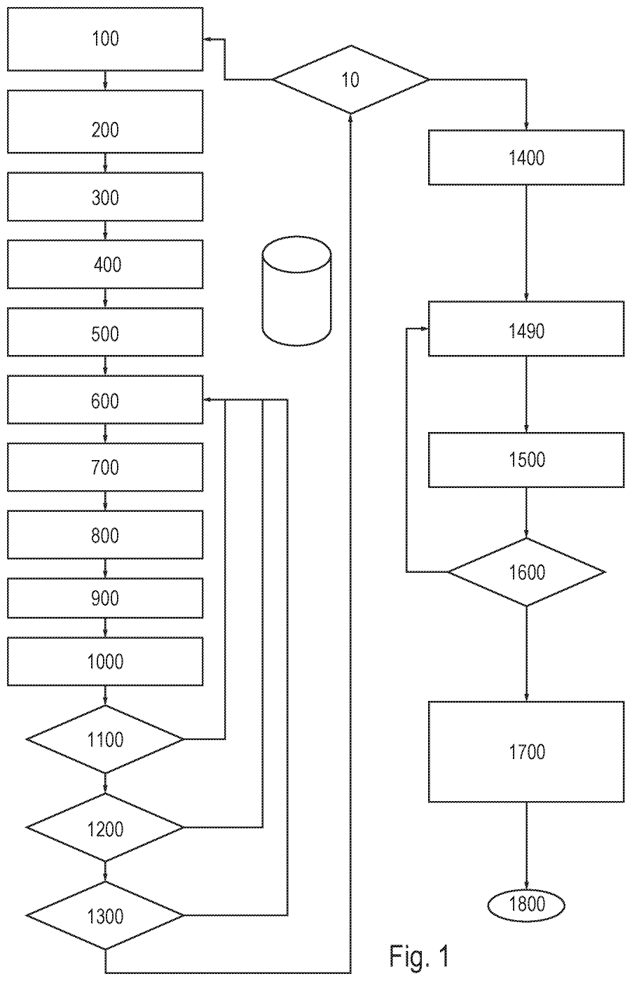

A first preferred embodiment (FIG. 1) according to the present invention relates to a flowchart indicative of a method according to preferred embodiments according to the invention.

The method starts in step 100. From an imaging device or preferably a data store comprising results of an imaging device, such as an MRI or CT scanner, information pertaining to such scan of a patient is received and processed to a patient specific heart/torso model. Alternatively, and already prepared heart/torso model is received. Alternatively, a model that best matches the body size indicated by a stretch sensor of an ECG patch is selected from a data store, which is disclosed below in greater detail. Also anatomical information obtained from a 3D camera image may be applied by selection of such model from a data store. Alternatively, a 3D location of the ECG electrodes may be obtained from placing the electrodes within a stationary or alternating electrical, magnetic, or electromagnetic field.

In step 200, information pertaining to, preferably all, intracavitary cardiac structures, such as papillary muscles or moderator band, are received for inclusion into the model. Positions, size and orientations of such structures may also be obtained through other cardiac imaging modalities, such as echocardiography. It is envisaged to estimate scar tissue if present. Preferably, such scans are based on MRI or CT data, but an estimation based on mecha-nical movements determined with an echo device are also envisaged.

In step 300, an estimation is made of the presence of myocardial tissue that may be not fully functional, which means of which the electrical function is hampered due to a lack of blood supply or due to other causes that have changed the electrical properties of such tissue of the myocardium. Probably the most frequent cause of a reduction in propagation velocity of an activation of such tissue is a previous heart attack. The result is the existence of areas of scar tissue, which may be indicated as nonfunctional myocardium, or ischemic tissue, in which the propagation velocity is reduced due to the limited blood supply to these cardiac cells. Areas with ischemic or scar tissue may be identified from MRI (delayed enhancement MRI) or CT imaging. If no such imaging data is available, the scar size and position can also be estimated through alternative methods, such echocardiography, radionuclide methods, or ECG.

In step 400, and identification is made of anatomical locations of the His-Purkinje system. Normally, the optimal electrical activation of the ventricles is produced by the His-Purkinje system originating from the AV-node. The stimulated CRT activation sequence should match the intrinsic cardiac activation sequence through the His-Purkinje system as close as possible to obtain the best cardiac function. In this functional block the anatomical locations of the His-Purkinje system activating the myocardium is identified using patient specific input from medical imaging information, such as MRI, CT, or echocardiography.

There is a practical constraint of reaching heart location based on the implant procedure chosen. Left ventricular endocardial pacing differs from e.g. left ventricular endocardial pacing. Frequently a CRT lead is positioned through one of the cardiac veins. Not every vein is equally accessible, and some might overlay scar area, from where the myocardial tissue activation may be limited or off-limits. To localize the accessible cardiac pacing space (e.g. veins, or left endocardial wall) prior or during the implant procedure is therefore preferable with providing that therapy guidance, and reducing the implantation procedure time use. The accessible cardiac pacing space may preferably be estimated or determined from x-ray angiogram imaging data recorded during the procedure, or preferably before the procedure. Accordingly, in step 500, such location information is received and/or processed.

Relating to step 600, the ECG is recorded, wherein the ECG is measured on the body surface and from the moment of insertion of pacemaker and/or the electrodes in the torso, also from signals from the electrodes. Such invasive ECG signals may preferably be used to optimize the heart model in step 700. Preferably, the positions of the electrodes on the chest are measured by means of the a 3D camera. Other per se known methods of recording the ECG location are envisaged in this context, such as identifying the location of the ECG electrodes within a stationary or alternating electrical, magnetic, or electromagnetic field.

Relating to step 700, a computational model is created or determined based on the model comprising cardiac geometry, cardiac structures, cardiac scar and cardiac conduction system, preferably as electrophysiological model. Such electrophysiological model relates to the electrophysiological processes within the heart and the myocardium. The patient specific electrophysiological model is used to determine or simulate a patient specific ECG. Fine tuning of internal electrophysiological model parameters is preferably applied to approximate the simulated ECG to the observed patient ECG.

Relating to step 800, areas in which cardiac pacing leads can be potentially implanted are determined. For guidance purposes not all possible cardiac pacing positions are suitable. For instance a cardiac pacing lead is preferably avoided in an area with scar. Lead placement through veins of the coronary sinus require availability of a cardiac vein. Similarly, endocardial pacing are considered.

Relating to step 900, for a number of potential cardiac pacing configurations, preferably indicated as locations of the possible cardiac pacing leads and the possible timings of the cardiac pacing leads, heart synchrony indices are determined. Systematically changing the position of only one cardiac pacing lead to all possible cardiac location (e.g. on the left ventricle), while keeping all other cardiac pacing leads, and cardiac pace timings constant, creates 3D synchrony guidance maps. Such synchrony guidance map assists to visualize the location where optimal synchrony is achieved. Initially, at the start of the implant procedure, no cardiac lead is implanted, and a synchrony guidance map will be created for a first lead. Iteratively, further leads will be added. With this, a position of the previously implanted lead is kept constant.

In addition, varying the pacing lead configuration to create a 3-dimensional synchrony map, timing of the electrical stimulation on any chosen electrode of the pacing lead is performed. Modification of electrophysiological timings, such as the PR/AV timing that may be controlled by a pacing system is part of the timing parameters that are varied. Determination of the cardiac synchrony map may be done independently for a lead configuration and for timing per lead, or for a combination thereof.

The number of such lead positions and timing parameters provide an equal number of synchrony maps to assist in the cardiac pacing therapy guidance. Such maps may either be individually displayed, or the optimal guiding synchrony map may be displayed by the system using pre-defined criteria or based on input by the person performing the implant. Examples for this comprise left epicardial, left endocardial, or right endocardial, wherein the optimal position is indicated for a preferred selected lead or targeted lead.

Relating to step 1000, feedback is provided during the implant to inform the physician whether the targeted cardiac lead position is reached. Confirmation of the current lead position can be generated by several cardiac mapping technologies, including the use of the VCG, an inverse ECG procedure, localizing the pacing spike. Additionally, location information for guiding the implanter may be input from another system, and may be displayed for the implanter together with the guidance and activation map. Such system is preferably magnetic or electric, 3 dimensional gradient field based localization systems. The location is displayed on the cardiac activation and synchrony map to assist navigation towards the target location. For such location the synchrony is preferably determined.

Relating to step 1100, a determination is made as to whether the target position is reached. This is a confirmation step: The implanter is supported by guidance technology to reach the optimal target location based on the chosen 3-dimension guidance map. Once the implanter has reached the final target location, the system request input such as by providing a prompt to confirm the achievable synchrony at the target location corresponds the predicted synchrony.

Relating to step 1200, the system request input such as by providing a prompt as to whether a further pacing system lead is to be implanted.

Relating to step 1300, the system request input such as by providing a prompt as to whether a further optimization of one of the previous leads is preferred in light of the current position of the lead, and/or to input a new target for any lead to be repositioned when this is preferred. When finished, information of the determined model, calculations, results thereof, placed lead locations and applied pacing system settings are preferably stored in a data store for later use as a set of data indicated as pacing system, such as CRT, implantation data or model, as well as ECGs.

Relating to step 1400, these patient data are obtained from the data store for performing improvements relating to the settings of the pacing system.

Relating to step 1500, a target synchrony is determined with optimal pacing system settings, preferably with the present electrode positions as implanted before. The CRT device is optimized using variations in AV delay, which may be indicated as a delay between atrial stimulus and the ventricular stimulus, and VV delay, which may be indicated as a delay between right and left ventricular stimulus.

Relating to step 1600, when the target synchrony is reached, system is optimized, the method returns to step 1490 which is substantially the same as step 600.

Step 1700

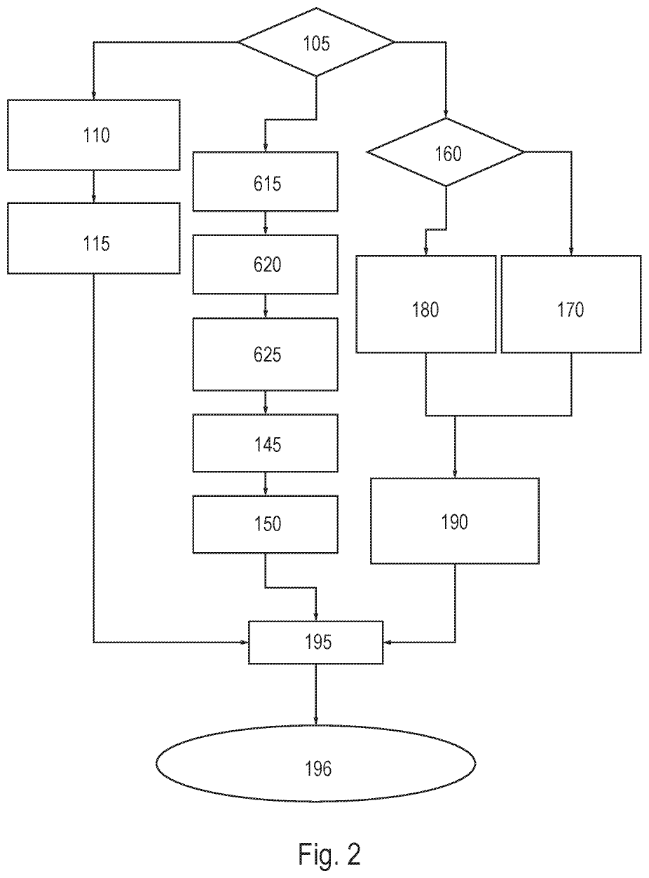

FIG. 2 shows a flowchart indicating details of step 100 according to a preferred embodiment. In step 100 the patient heart/torso model data is received and/or processed. In step 105, it is determined which imaging modality is available to create a heart/torso model. In step 110, relevant medical imaging data, such as MRI, CT, PET, echo etc. are received based on which the reconstruction of the heart/torso model is to be created.

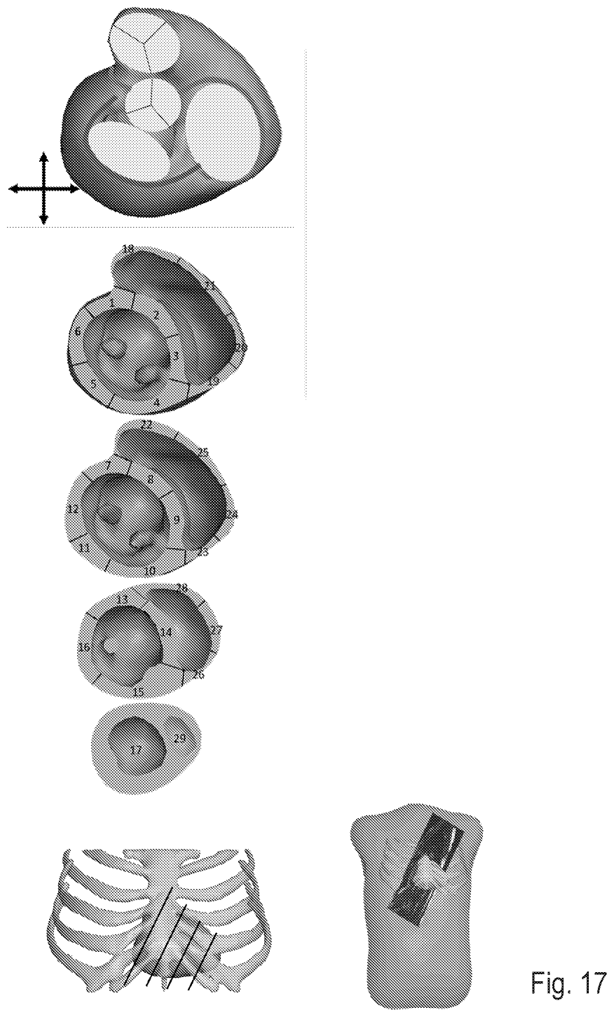

Distinct data modalities have different capabilities to construct a model of the patient. A heart model is selected from a model database determine the contour of the tricuspid valve, aortic valve, the mitral valve and eventually the pulmonary valve. Also localize the left apex. Based on this information a model the best fitting model can be selected from the database in step 115. MRI, PET, and CT may be used to reconstruct the heart and torso geometries. Echo and Rotational x-ray may be used to reconstruct the heart. The 3D position of the heart is preferably performed by using a 3D camera to localize the echo probe and to reconstruct the torso surface. An example is shown in FIG. 17.

In step 145, a 3D image with analyzed body parts is used to reconstruct the surface of the thorax. As the 3D photo(s) often lack data of the back of the patient, this part of the thorax is interpolated such as by using the location of the table the patient is lying on. Based on such data ellipses may be fitted between the part of the thorax that is visible and the part that is not visible. This first estimated thorax model may be used in the next step to select the optimal model.

In step 160, a heart/torso model is selected from the database that best matches the reconstructed torso model from a 3D regarding, based on size chest circumference, gender and if available age of the patient. Subsequently the selected torso model is adapted to match the parts of the thorax captured in the photo accurately. As left and right of the body are similar, parts of the body may be reconstructed using the mirror symmetry.

In step 190, a heart/torso model is selected based on metadata. In case a stretch patch was used the chest circumference may be used. The algorithms deliver a model that matches the patient's chest as a best fit.

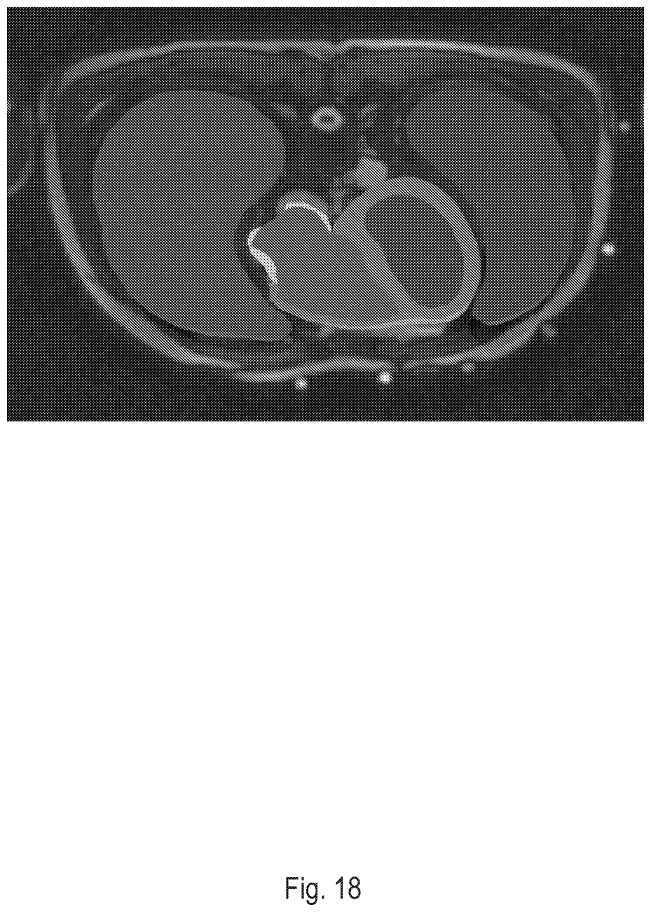

In step 195, the resulting model of torso with heart for the patient is displayed to provide a visual inspection of the reconstructed or selected model, see heart with blood cavities merged with an MRI image of FIG. 18.

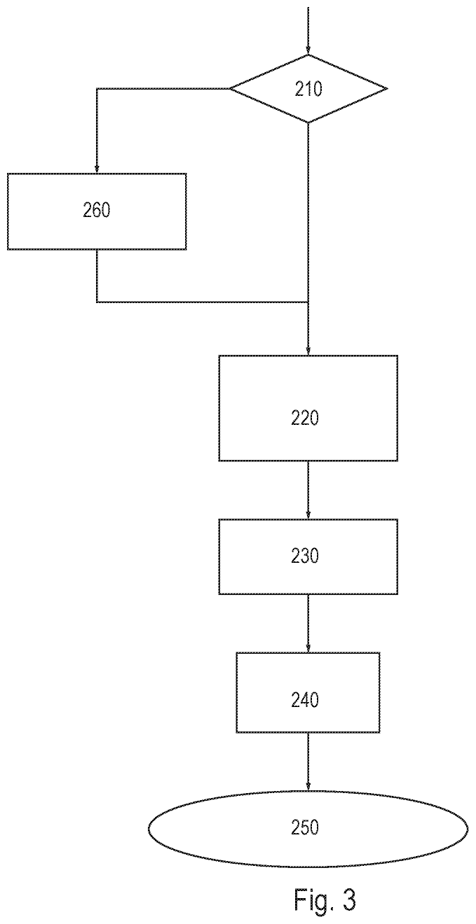

FIG. 3 shows a flowchart relating to the steps comprised by step 200 according to a preferred embodiment.

In step 210, it is determined which imaging data is available, such as MRI or CT, Echo or X ray. In step 220, the intracavitary structures, papillary muscles and moderator band for instance, are patient specific. That means that they are attached to the cardiac wall at patient specific positions. Any of the imaging modalities that were be used to localize these structures may be applied here. Preferably a direct merge of the model data and the imaging data is performed, when possible. When for instance a stand-alone echo machine is used, the automatically detected papillary muscles can be manually matched to the a segment of the heart. The identification of the papillary muscles may be identified by a line from the base of the papillary muscle, such as connected to the myocardial wall, to the tip. For a moderator band the connecting line may extend between right septal wall and right free wall.

In step 230, the identified intracavitary structure is incorporated into the (computer) heart model. In step 250, in case a no imaging data was available and a standard heart model is used, the intracavitary structures may be identified using other imaging modalities. This is performed with matching of the heart model to the used imaging modality. An example of such matching, process is given below. In step on the 60, it is checked whether an alternative thorax size sensor based on the ECG electrode system attached is available. In step 170, an alternative thorax size sensor based on the ECG electrodes attached is used. One implementation of such a sensor is a special (multi-electrode) ECG electrode patch, with stretch and/or bend sensors to measure the thorax size and the chest circumference available. Another implementation may be to use an impedance measured between different electrodes of the ECG system.

In step 180, such as when no alternative thorax size sensor is available, the thorax size will be estimated from clinical patients characteristics, such as height, weight, gender. In step 190, a heart/torso model is selected based on metadata available. In case the stretch patch is used the chest circumference may be used. The algorithms preferably delivers a model that matches the patient's chest as a best fit.

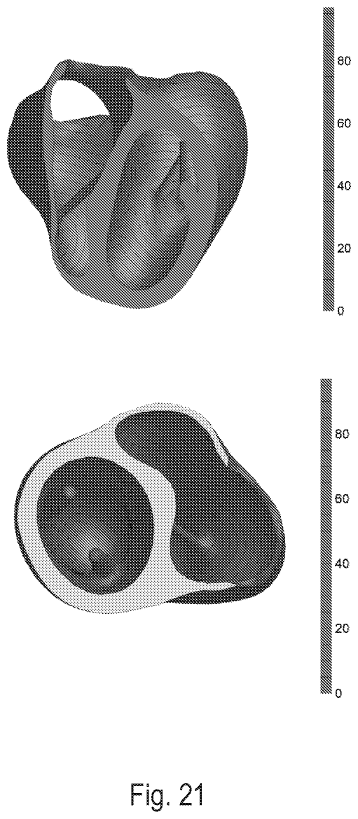

In step 195, the 3D heart torso model combination is shown as shown in FIG. 21. This model is also used in other functional steps.

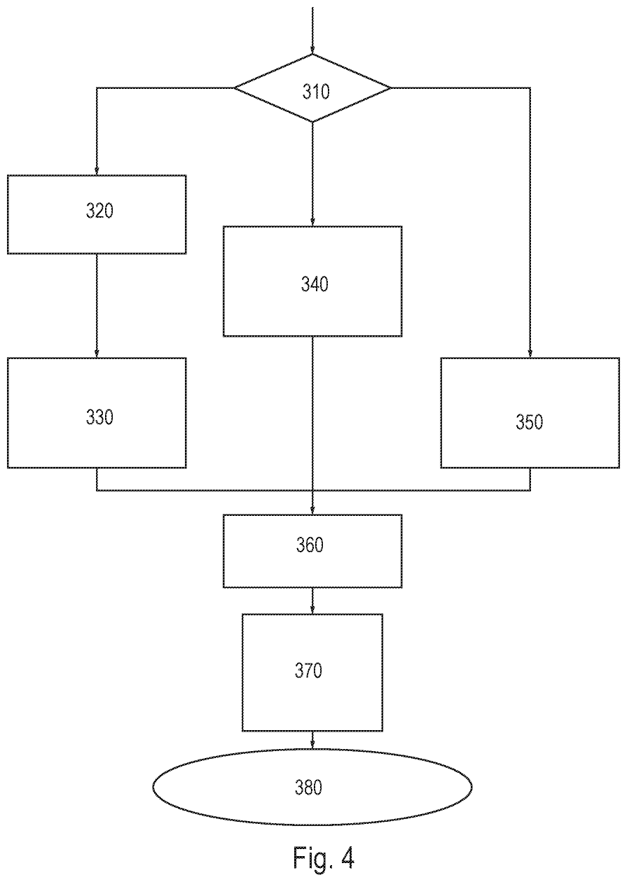

FIG. 4 shows a flowchart relating to the steps comprised by step 300 according to a preferred embodiment.

In step 310, it is determined which imaging data is available, such as MRI/CT or Echo or X ray. In step 320, in case no imaging data was available and a standard heart model is used the intracavitary structures may also be identified using other imaging modalities, this requires the registration of the heart model to the used imaging modality. An example of this registration, process is given below. In step 330, scar tissue is localized, such as from Angiogram data. The less perfused areas may be identified and used as a measure for the localization of ischemic/scar areas. From echo the less or delayed activated areas identification may be used to localize scar/ischemic areas.

Step 340 relates to Scar localization. Scar tissue is non-viable myocardial tissue that may be identified using a specialized MRI or CT sequence, or alternatively through echo or other imaging options. For MRI a preferred sequence is known perse as delayed enhancement MRI. The algorithm preferably analyses the medical images on specific coloring within the myocardial tissue. For the delayed enhancement MRI the areas with non-viable myocardial tissue has a light color, which is identified as scar.

350: Scar localization: In case no medical imaging is available to identify scar the scar tissue is preferably estimated using other methods. For instance, the Selvester score 1 is a method based on the ECG (the baseline ECG can be used) to identify regions of scar. Scar can also be detected using echo images or other imaging modalities.

360: Use the identifies scar/ischemic areas to adapt the heart model so it can be used to estimate the local propagation velocity based on the classification of the myocardial tissue, e.g. healthy, ischemic, or scar.

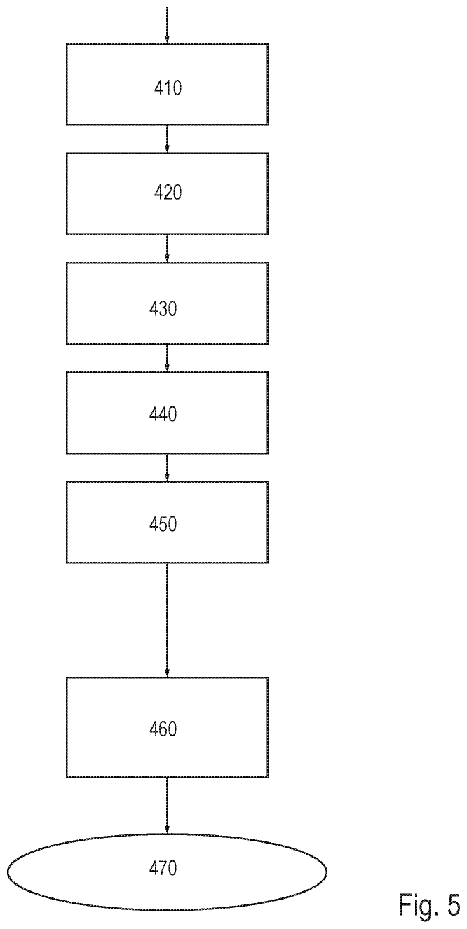

FIG. 5 shows a flowchart relating to the steps comprised by step 400 according to a preferred embodiment.

410-450: Compute 6 distinct nodes from the heart geometry as the starting points for the His-Purkinje activation 3. Given known valve nodes, the papillary muscles nodes and moderator band nodes, the following nodes positions may be computed:.
The base of the left and right papillary muscles (410) and right free wall side of the moderator band (420)
Right septal area, about 1.5 cm from the right apex (430)
Left septal wall close to the mean ventricular wall mass (440)
Left septal wall close to the aortic base (450)

The first node to activate is the left septal wall, followed by the rest at about 10-20 ms, depending on the distance between the AV node below the RCA entry of the aorta and the respective identified node.

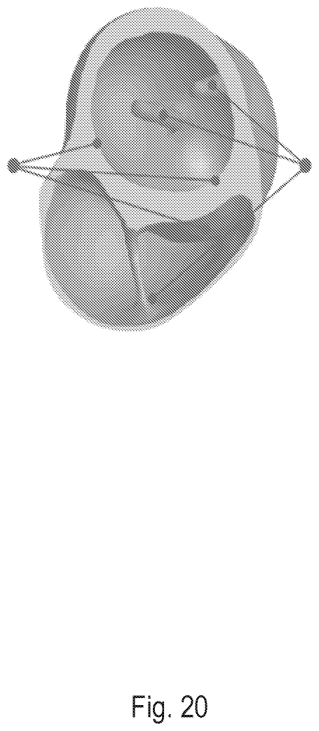

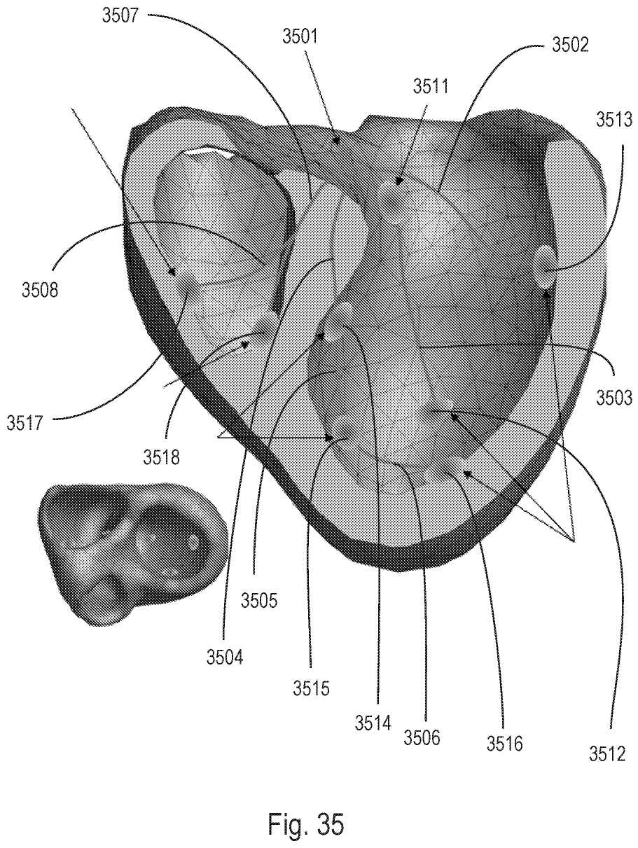

460: Display the His-Purkinje system in the model as in FIG. 20 or 35.

A representation of a subject specific CT-based model with the six regions associated with early ventricular activity in during normal cardiac activation e.g. sinus rhythm. Three structure foci or endings, two left ventricular papillary muscles and the moderator band were identified based on cardiac imaging. Three septal regions were automatically identified based on anatomical landmarks in the ventricular model. Septal and structure foci were identified based on knowledge from His-Purkinje anatomy and regions of early cardiac activation (Durrer, Tawara).

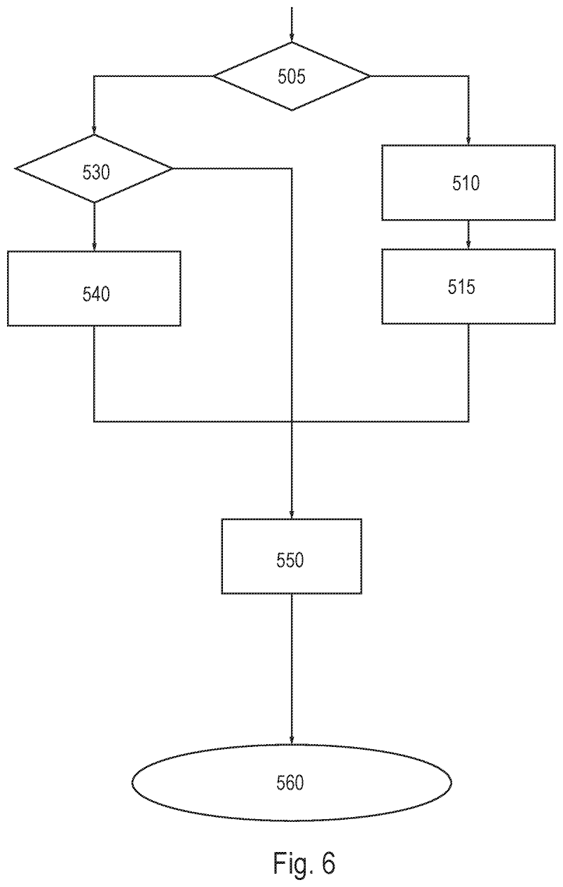

FIG. 6 shows a flowchart relating to the steps comprised by step 500 according to a preferred embodiment.

505: Determine if angiogram data is available for this patient, data with which the left ventricular veins may be identified. A patient specific model derived from MRI or CT preferably improves the quality of the registration of the angiogram data with the heart/torso model.

510: Segmenting of the left ventricular veins using CT is rather preferred. The veins are preferably followed through contrast.

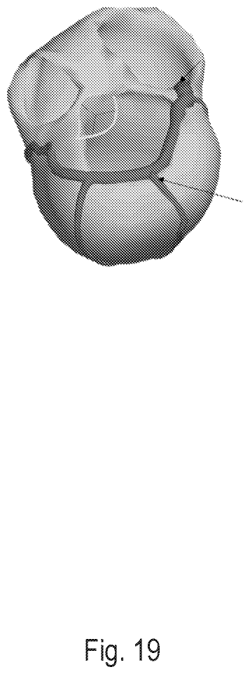

The veins may be reconstructed from X-ray data (as exemplified in FIG. 19)

515: The reconstructed veins preferably fit the heart model, but might be slightly off due to registration errors. The model and reconstructed venous system are optimally matched to support the guidance of the LV lead placement.

530: Enough data available to estimate the venous system?

540: The venous system may be estimated based on the cardiac anatomy. The position of the coronary sinus (CS, exemplified in FIG. 19), the end of the cardiac venous system, may be anatomically determined. Also the branches over the myocardial wall may be estimated using the geometrical/anatomical identification of the coronary sinus landmarks. An example is shown in FIG. 19.

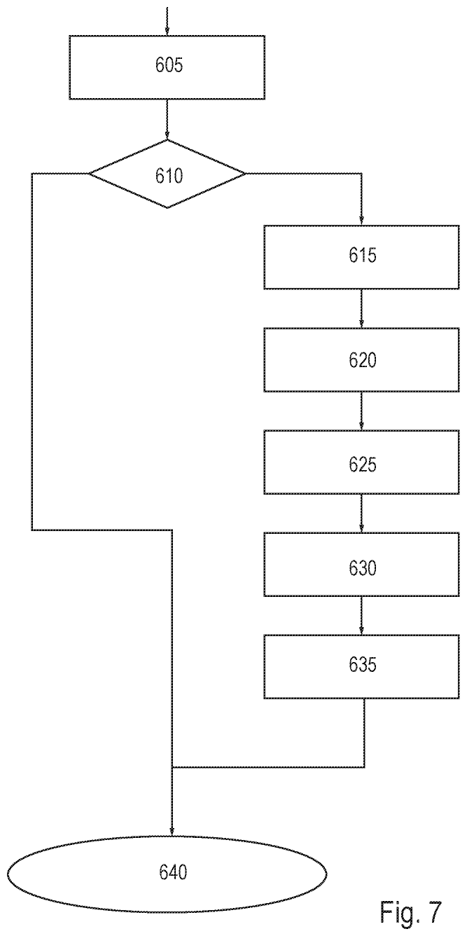

FIG. 7 shows a flowchart relating to the steps comprised by step 600 according to a preferred embodiment.

605: Obtain the ECG, for example by receiving ECG data. For the standard 12 lead ECG this the left and right arms, the left foot and the precordial leads V1-V6 over the heart as shown below. Other signals from catheters in the heart (e.g. the pacemaker leads) or signals from other measurement devices, blood pressure measurements etc.

610: Is a 3D camera available to localize the ECG electrodes on the chest? If yes, 615: Record the 3D photo using a 3D camera, and subsequently, 620: Detect the fiducial markers on the 3D image, if available.

625: Analyze the 3D photo and localize and characterize the different body parts (see 100 thorax model)

630: The ECG cables and electrodes are specific per manufacturer. Shape and colors are different. To preferably localize the electrodes automatically these electrode/cable connector features are preferably retrieved from a database.

635: Localize the ECG electrodes using the shape and color features of the electrodes and ECG cable used. The features in the photo that match database features are used to find and localize the specific ECG electrodes on the chest

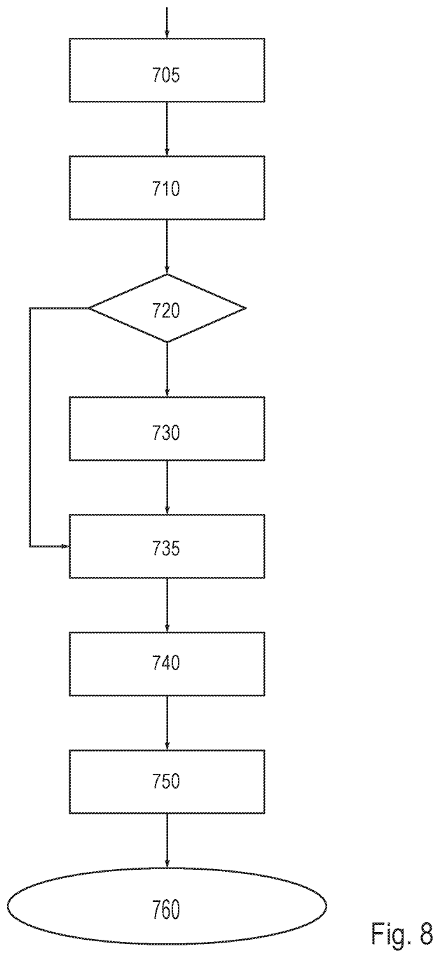

FIG. 8 shows a flowchart relating to the steps comprised by step 700 according to a preferred embodiment.

705: Compute the internal distance matrices according to van Dam PM, Oostendorp TF, van Oosterom A. ‘Application of the fastest route algorithm in the interactive simulation of the effect of local ischemia on the ECG’. Med Biol Eng Comput 2009; 47:11-20. This latter matrix (S) contains preferred connections over the myocardial wall and through the myocardial wall of the 3D model (100).

710: Assume a homogeneous propagation velocity over the wall of the myocardium and about 2.5 times slower perpendicular to the wall to compute the velocity matrix (see anisotropic ration van Dam et al 4).

720: When scar (300) is present the propagation velocity from node to node in the 3D model (100) is preferably adapted

730: In areas with scar the cardiac activation propagates slower or even not at all. In the areas connected to areas with died myocardial tissue the propagation velocity is reduced whereas in the full scar (dead myocardial tissue) the velocity is 0, the activation stops. CRT patient have suffered frequently from a heart attack and therefore have areas with scar tissue

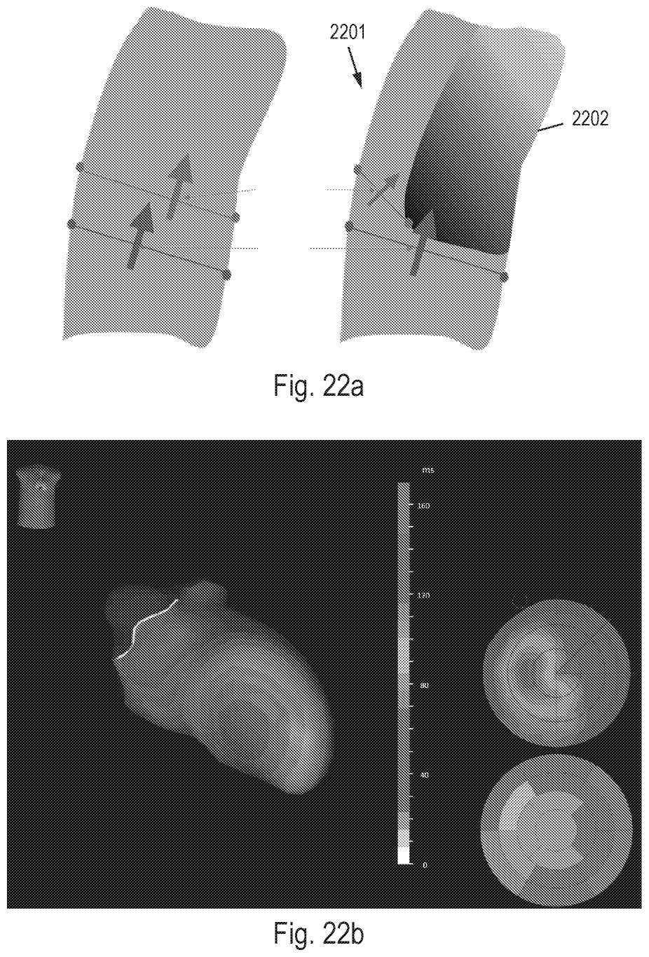

Therefore the identification of scar is necessary in preferred. For healthy tissue a normal propagation velocity is 0.8 m/s whereas in the border zone the velocity diminishes, e.g. toward 0.1 m/s, and is 0 m/s in the full scar area. An example is shown in FIG. 22A.

For each of the connections identified in the adjacency matrix 4 a propagation velocity is estimated (V).

740: Using the matrix V and S enables us to compute the heart timing adjacency matrix T=S/V. Using the fastest route algorithm enables the computation of whole activation sequences for the whole heart from any node to preferably all other nodes.

750: Based on the His-Purkinje foci and the timing matrices compute the normal activation and the scar related activation (preferred for this patient with conduction system)

760: Display the estimated activation map. Below a few ways to display this map. The certainty can be computed as the match between the simulated ECG from this activation map and the measured ECG. From the map several parameters can be derived: the total activation delay in the left ventricle (LV, figure left panel), or the standard deviation tin the LV timing. An example is shown in FIG. 22 B.

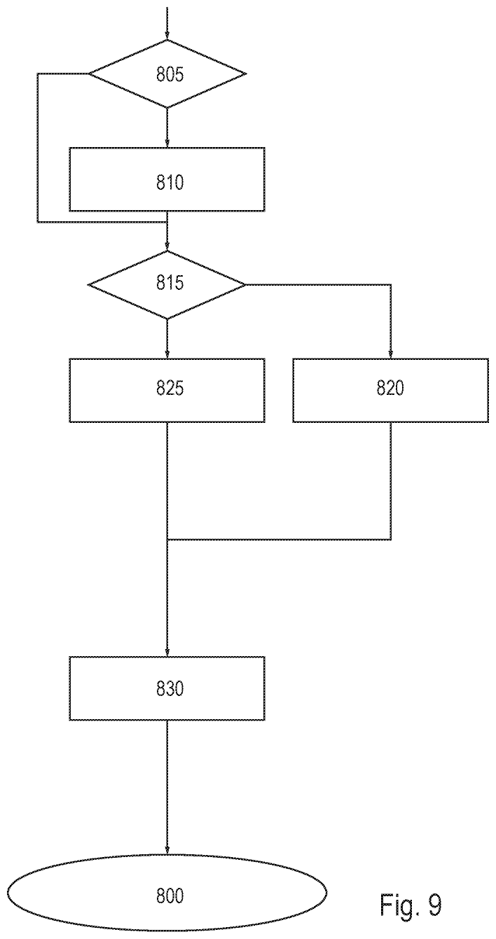

FIG. 9 shows a flowchart relating to the steps comprised by step 800 according to a preferred embodiment.

Initially the LV lead could be positioned at will. Scar and the availability of veins limit the search area.

805: Is scar defined and present in the analyzed data (200)?

810: Exclude the areas with scar from the target search area for lead placements (combine 100/200). This is a simple overlay of the determined scar area with the cardiac anatomy

815: Is this a LV epicardial lead implantation?

820: The LV lead is placed endocardial, so exclude the LV epicadial area from the potential lead position area.

825: Exclude epicardial LV areas where no vein is present or the vein is most probably not accessible by a guide wire as determined in 500. This latter conclusion is preferably done on veins reconstructed from medical images. When estimated veins are used a larger distance margin to the vein position may be used.

830: Show potential target areas on the 3D model (100) to support the CRT implant procedure

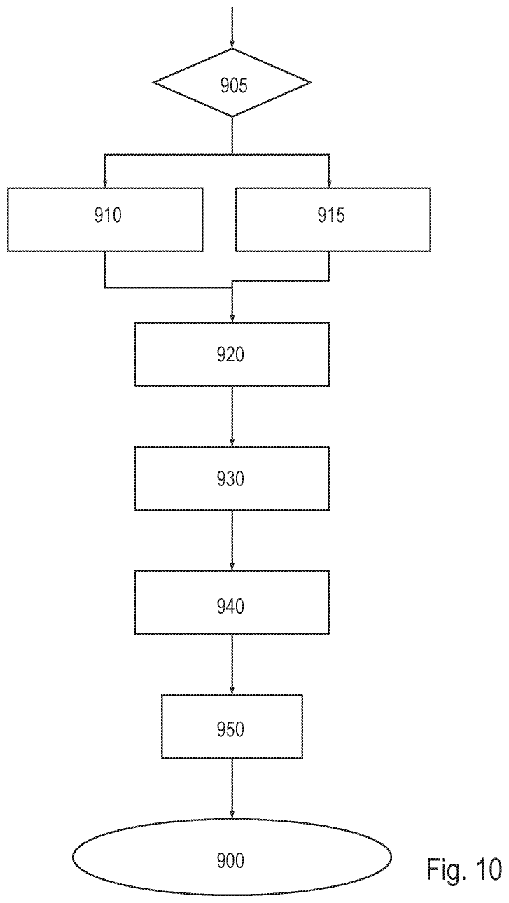

FIG. 10 shows a flowchart relating to the steps comprised by step 900 according to a preferred embodiment.

905: What is the current target lead, RV lead or LV lead?

910: Exclude possible locations in the RV area for RV target lead (800)

915: Exclude possible locations in the LV area (epi and endo) (800)

920: Based on the excluded areas from 400 and 510 or 515 preferably a limited number of areas are available to implant a pacemaker lead. When multiple leads are preferred to be implanted a combination of areas is selected as potential lead locations. When all leads have been implanted there are preferably two positions for which the synchrony can be computed.

930: Predict the synchrony using the current determined first lead location with the second lead location. The synchrony may be based on technology described in the pct/ep2016/066424, but also the comparison to the His-Purkinje activation or using an electro-mechanical model, e.g. CircAdapt.

For this prediction maps per node may be computed. A prediction map is computed per node of the ventricular geometry, and gives a prediction in the potential improvement of cardiac function, e.g. cardiac output, mechanical work etc. The prediction number preferably translates a certain parameter value into a number between −100-100, where −100 produces potential the opposite effect (worsen sunchrony), and 100 is optimal for the given parameters. The prediction maps are preferably computed using the activation map (700) combined with an added stimulus from any of the ventricular geometry nodes (100-500). Based on this simulated activation sequence the ECG may be computed. Both ECG as well as activation/recovery times may be used to estimate the change in any of ECG/activation/recovery times derived parameter.

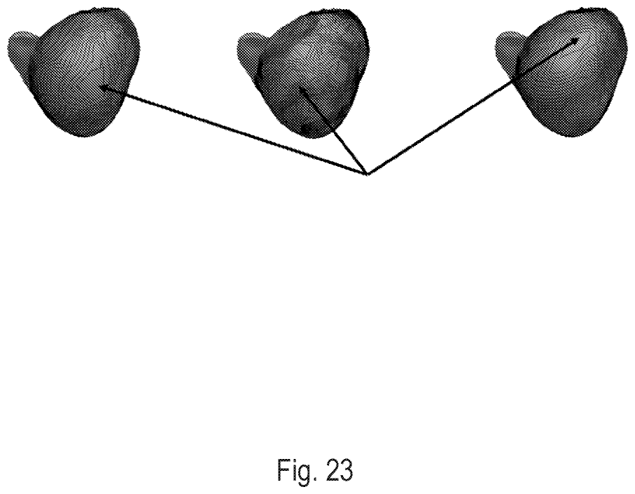

Preferred prediction parameter maps that can be computed are shown in FIG. 23.

The QRS area may be computed according to Prinzen et al, both with a KORS transform and one with the meanTSI transform (vector patent). The 3D meanTSI method uses the ECG to transform it into a VCG (vector cardiogram) signal, while using the center of the vector loop (VCG) and computes the area of the VCG with this center point. To compare the areas, the VCG is normalized to the maximum VCG amplitude, i.e. VCG amplitude is between 0 and 1. Blue shows a preferred QRS area in the QRS area maps below, red predicts a worse outcome. The maps 11, 13 and 14 predict all a similar region. An example is shown in FIG. 23

Mean TSI distance (9) Trans-cardiac ratio (15) and LV meanTSI ratio (17)

The 3D distance between beginning and end of the meanTSI path (colored line, red start, purple latest activation) is a measure of the synchronicity of the activation of the heart.

This mean TSI distance can be normalized for heart size to obtain a relative number in %. Resulting in a trans cardiac ratio (15) or LV mean TSI ratio 17 (see below, blue is high prediction, red low)

Normalized with QRS duration: Mean TSI distance (9), (red is high prediction, blue low)

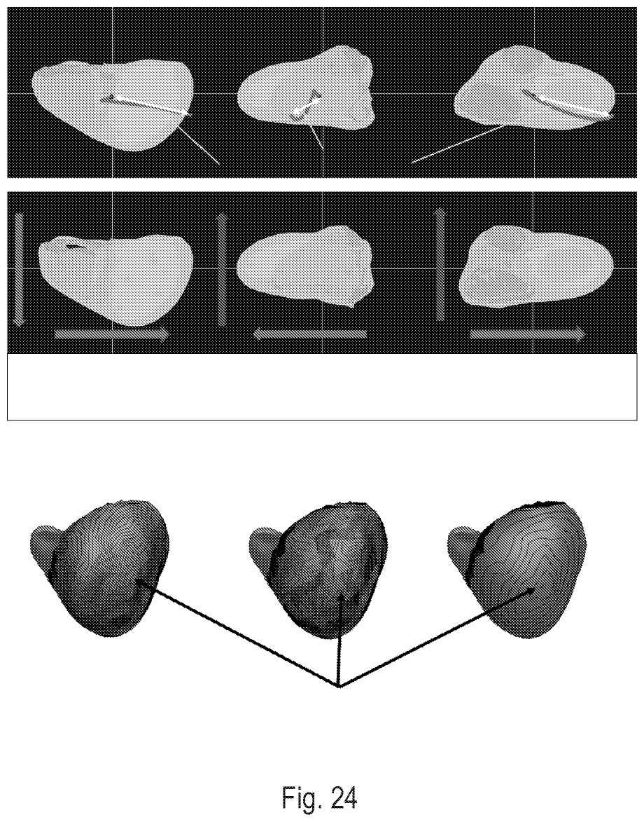

Small numbers are usually found in His-Purkinje activations and incomplete right bundle branch blocks. Large numbers signify an asynchronous activation. An example is shown in FIG. 24.

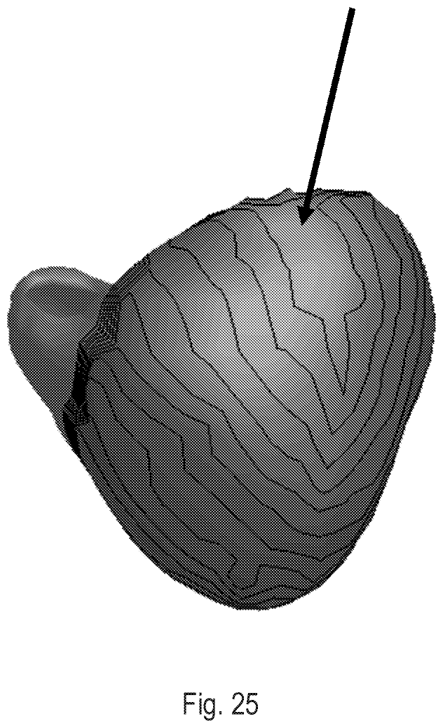

Shortening of QRS area, green shortens the activation time most. Adding a stimulus at this position shortens the QRS most. An example is shown in FIG. 25

Predict optimal papillary muscle timing: Papillary muscles are preferably activated close to simultaneous to prevent mitral regurgitation

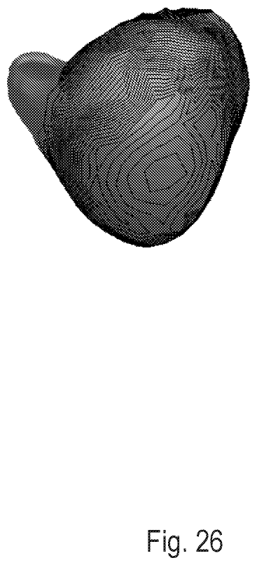

Parameter: [symbol] (t [symbol] _lateral-t_septal)+( [symbol] (t [symbol] _lateral+t_septal))/2
This parameter is large when the activation time t is late or when the difference is large. Small values are optimal. An example is shown in FIG. 26.

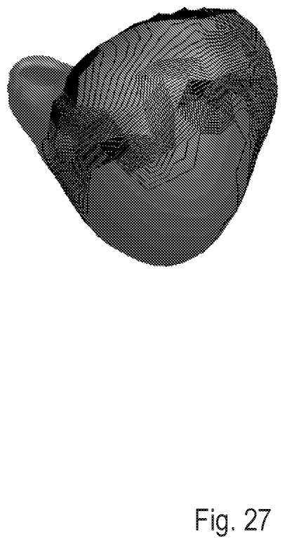

T-wave direction: The T-wave vector direction derived from the mean TSI or VCG is in normal subjects along the septum from base to apex, which might indicate the heart also mechanically recovers base to apex generally. A more optimal recovery is therefore also more when the T wave vector is directed from base to apex. An example is shown in FIG. 27. The latest area is for this parameter less optimal (green/blue area)

940: Determine the target position for the target lead, i.e. the position that gives the highest predicted synchrony (930). As every prediction map gives a number between −100 and 100, a combination of the most predictive maps can be used to select the optimal lead positions. Combinations may be made by multiplying the prediction value A of a node with prediction value B of the same node. The prediction value P for any nodes i can be described as

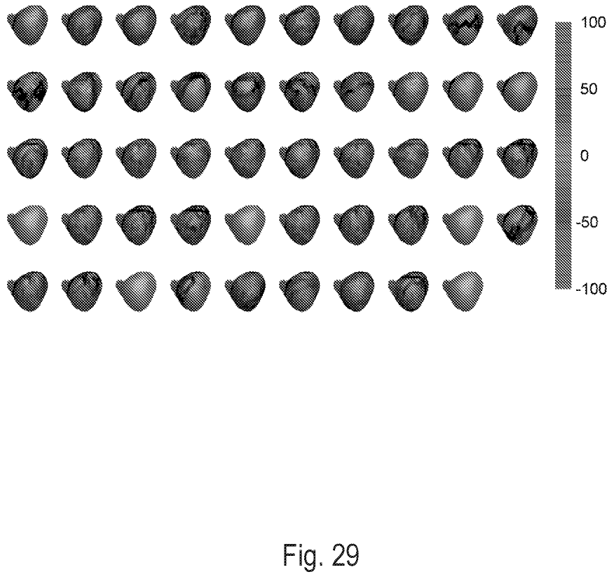

Pᵢ^{total}=Pᵢ^{QRSshortening}·Pᵢ^{QRS area}·Pᵢ^{mTSIratio}·Pᵢ^{LVAT}·Pᵢ^{xxx}

Where xxx is any other prediction map. Any other combinatory function on the prediction maps can be used, based on the appropriateness of the prediction map. An example is shown in FIG. 29

950: Display the location on the heart model (100) where the optimal position for the selected lead can be implanted.

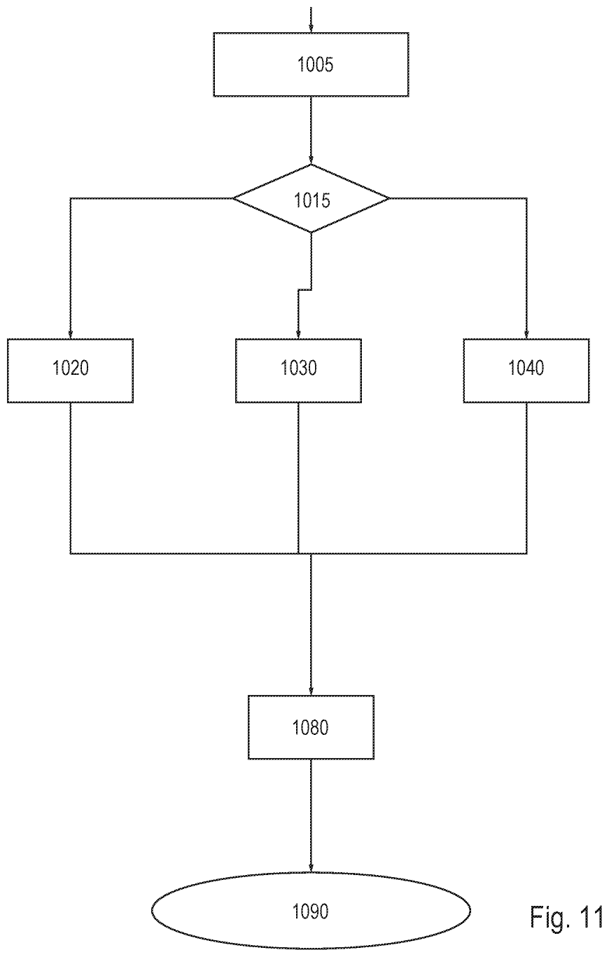

FIG. 11 shows a flowchart relating to the steps comprised by step 1000 according to a preferred embodiment.

In step 1005, a position the first CRT lead to the indicated location is received from 500. In step 1015, the technique to be used to localize the CRT lead in the heart model is determined. In step 1020, the mean QRS axis is used to localize the stimulus location according to pct/n12016/050728 as cited in the above. In step 1030, the simulated VCG to compare it with VCG from simulated isochrones taking into account the scar is used, also according to pct/n12016/050728 as cited in the above. . In step 1040, when a pacing spike is highly dipolar (little vector), the model of the torso and heart as a volume conductor model for the location can be estimated using a non-linear estimation procedure is used.

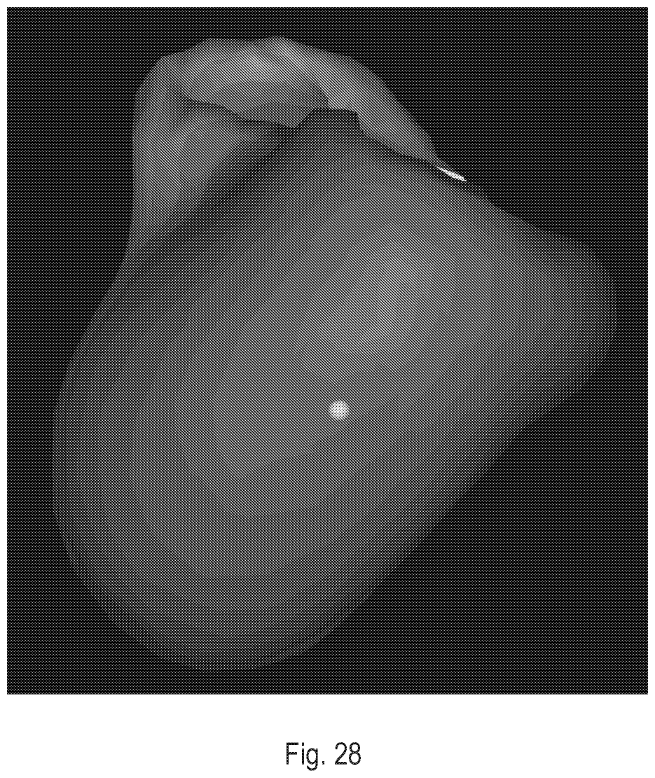

In step 1050, it is determined whether the target location is reached. In case it is reached, the next lead is placed with recomputed synchrony based on current lead position. In case it was not reached, the lead is repositioned. In step 1080, the localized lead position is displayed in the 3D model. The embodiment ends in step 1090. An example is shown in FIG. 28

An important element related to embodiments is whether the position of the lead is correct. To do this, the pacemaker may be activated to test, with which action, the location of the stimulus may be determined with use of pct/n12016/050728 as cited in the above. It is further preferable to take into account that distance is kept from scar tissue in light of a risk of hampered activation near such scar tissue.

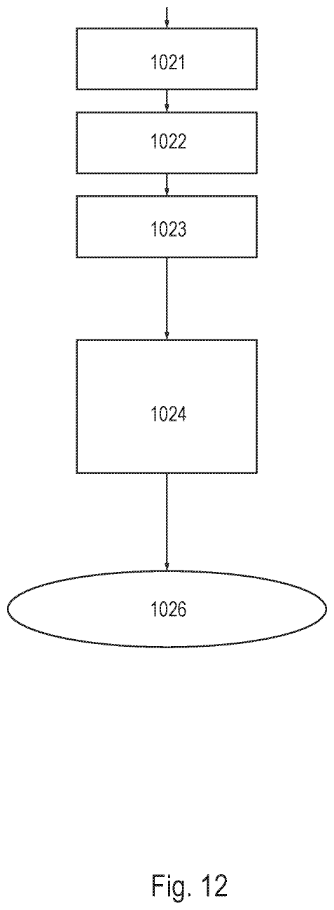

FIG. 12 shows a flowchart relating to the steps comprised by step 1020 according to a preferred embodiment.

In step 1021, the stimulated QRS is selected from the recorded ECG, of which an example is shown in FIG. 32.

In step 1022, the mean QRS axis is derived from the Vector cardiogram (VCG), computed as described by van Dam, PM. ‘A new anatomical view on the vector cardiogram: The mean temporal-spatial isochrones’, Journal of Electrocardiology 2017; 50: 732-8, of which the contents are herein incorporated by reference. 5. The VCG derived from the ECG provides reference to the electrode positions. A VCG is computed from the 9 electrodes positions, building the 12 lead ECG by:

$$\vec{VGG} ( t ) = {\sum }_{el = 1}^{9} {ecg}_{el} ( t ) \cdot \vert \vec{r} \vert \qquad eq . 1$$

where \vec{r}=|\vec{rₑₗ}−\vec{r_{ref}}| is the normalized vector between a cardiac anatomical reference position and the electrode position on the thorax and R the length of r \vec{ }. The [symbol] ecg [symbol] _el (t) is the value of the ECG on electrode at sample t. The VCG represents the direction of activation in the 3 principal directions, x,y, and z.

In step 1023, of VCG samples or all the VCG samples are added or summed over the duration of the QRS complex to preferably provide a mean direction of activation, i.e. the mean QRS axis. In this study the mean QRS axis is intended to preferably cross the center of ventricular mass (CVM).

In step 1024, a preferred, preferably computer implemented, algorithm subsequently determines where this positioned mean special QRS axis enters the ventricles, such as comprising the myocardium or valve planes of aorta, mitral, tricuspid and/or pulmonary valve. The number of cross sectional points is preferably varied between 3-6, such as twice for each wall. When the number of cross sections between the point of entry and the CVM is for example larger than 2, the algorithm is arranged to move the estimated stimulus location to the next cross section, such as from right free wall to septal wall for example when the initial 40 ms of the VCG changes direction or moves away from the mean QRS axis. The endocardium or epicardium is preferably related to an implant procedure of a respective CRT lead.

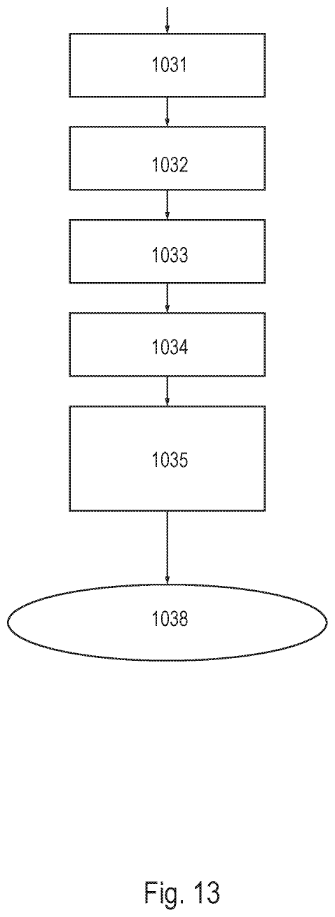

FIG. 13 shows a flowchart relating to the steps comprised by step 1030 according to a preferred embodiment. These steps are generally performed according to the above cited application pct/n12016/050728, generally indicated as follows. In step 1031, the stimulated QRS is selected from the recorded ECG (also in reference to step 1021). In step 1022, the ECG based QRS is converted to a VCG. In step 1023, the mean QRS axis is determined followed by a determination of an area of origin in relation to the mean QRS axis and/or the QRS mean TSI, preferably to provide a localization result in step 1024.

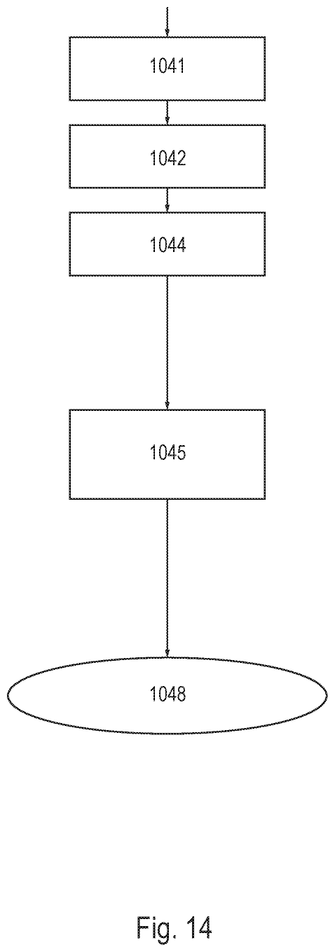

FIG. 14 shows a flowchart relating to the steps comprised by step 1030 according to a preferred embodiment. In step 1041, the stimulated QRS is selected from the recorded ECG (also in reference to step 1021). In step 1042, a pacing spike from the QRS ECG is determined, such a by extraction.

In step 1044, a transfer matrix is determined, such as based on the geometry aspects of the heart, thorax, preferably including other organs like lungs and liver, which is preferably used to convert a dipole at a certain position to potentials on the thorax surface. Based on measured spike potentials, a best fit is preferably created for a dipole on the heart surface. A pacing spike amplitude as delivered by the heart stimulator is preferably used as an additional parameter for a fit of the dipole.

In step 1045, such dipole is preferably applied as a source model for the pacing spike. Together with a 3D (such as a volume conductor) model (100) potentials arising from a dipole can be simulated at any position on the body surface. With preferably information of electrode positions (600), and the 3D model, a position of a dipole can be determined on the surface of the myocardium to simulate such potentials on the body surface at the ECG electrode positions. These simulated ECG signals can be compared to the measured ECG signals to preferably achieve information relating to whether such provide a better match than another comparison. It is advantageous to determine a best match.

The dipole on the node of the ventricular geometry for which the spike potentials on the thorax that provides the best fit to the measured potentials is selected as the current lead location. The embodiment ends in step 1048.

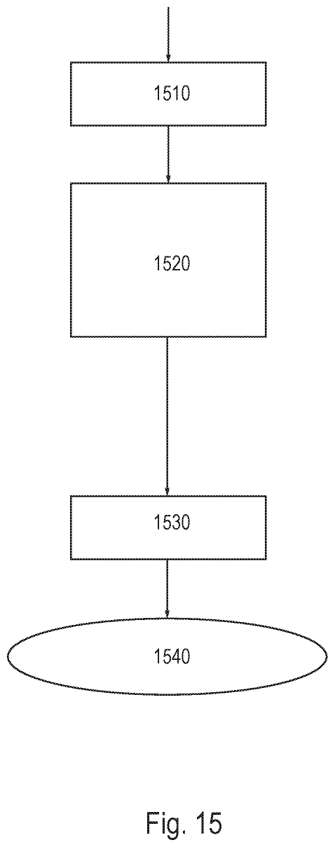

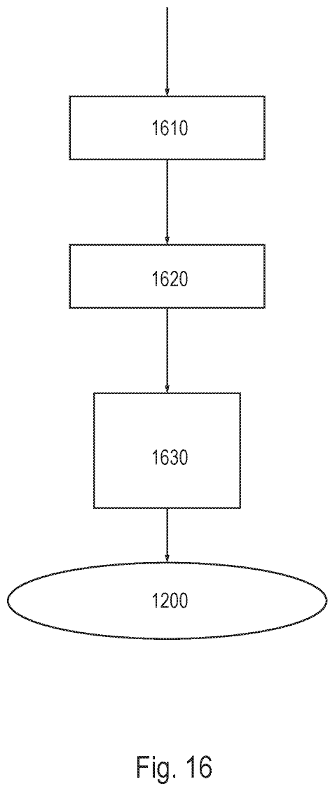

1400 obtain CRT implantation data from a database 11. Model (with scar etc (100-500). Used Lead placements (1300). Previous ECGs. FIG. 15 shows a flowchart relating to the steps comprised by step 1030 according to a preferred embodiment.1510: Based on number of electrodes on a pacemaker lead that can be used to stimulate the heart other electrodes can be selected for optimal stimulation. When preferably all leads have been implanted there are a preferred combination of these electrode positions available for which the synchrony can be computed. Determine the combination of electrodes to be stimulated. These maps are similar to the prediction maps, wherein the activation maps and measured ECGs are to compute the parameter values used in the prediction maps, so preferably a value instead of a whole map (1000).

1520: Predict the synchrony using the current determined first lead location with the second lead location. The synchrony may be based on technology described in the synchrony patent, but also the comparison to the His-Purkinje activation or using a electro-mechanical model, e.g. CircAdapt

As an example, with the varying parameters below
Test 1:
Atrial stimulus at 800 ms interval
Give LV stimulus at the distal tip of the LV lead at 90 ms
Give RV stimulus at the 100 ms
Test 2:
Atrial stimulus at 800 ms interval
Give LV stimulus at the distal tip of the LV lead at 70 ms
Give RV stimulus at the 100 ms
Test 3:
Atrial stimulus at 800 ms interval
Give LV stimulus at the distal tip of the LV lead at 50 ms
Give RV stimulus at the 100 ms
Test 4:
Atrial stimulus at 800 ms interval
Give LV stimulus at the distal MID of the LV lead at 70 ms
Give RV stimulus at the 100 ms
Test 5:
Atrial stimulus at 800 ms interval
Give LV stimulus at the distal PROXIMAL of the LV lead at 70 ms
Give RV stimulus at the 100 ms

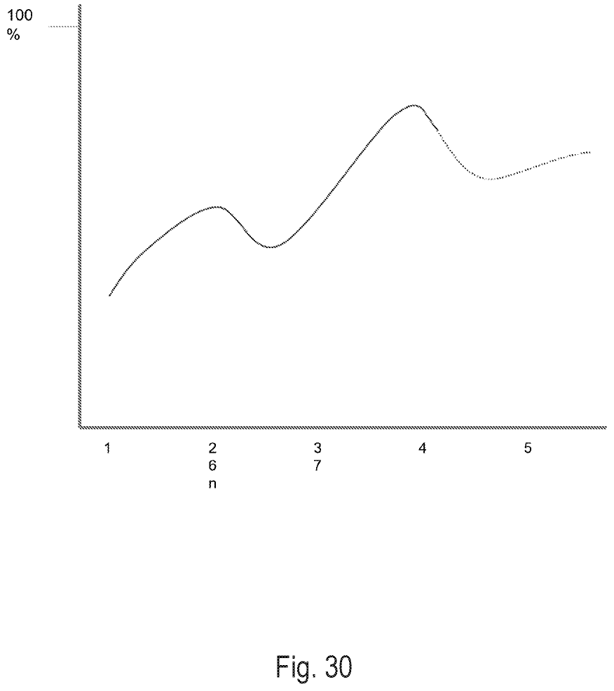

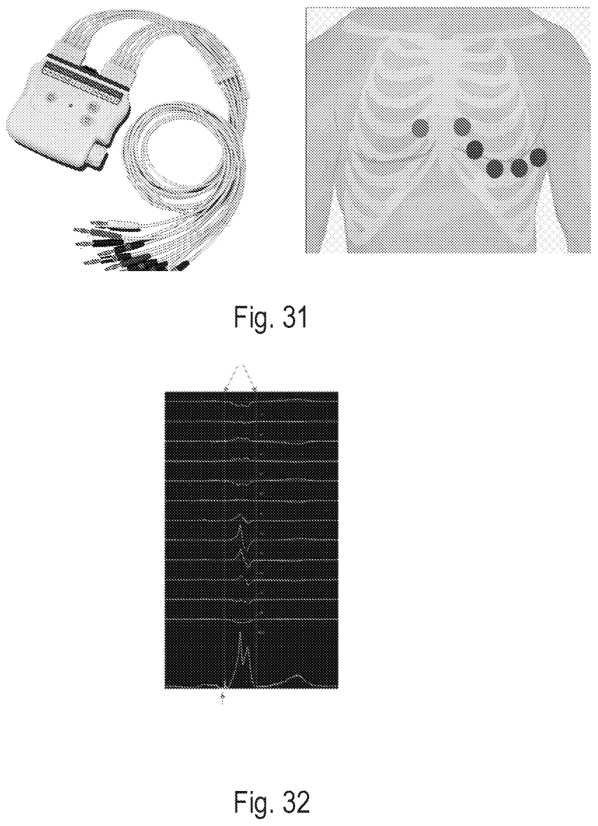

1530: Per test the synchrony may be determined and displayed, of which an example is displayed in FIG. 30.

1500 Have all tests been performed (output of 1500)

Change CRT settings

Change the setting of the CRT device, with the optimal settings as determined through the test. For the example below the settings selected ar eteh setting for test 7
The timing parameters AV delay and VV delay
The stimulated electrodes or nr of stimulated electrodes

Further preferably, in step 1700, a pacemaker is programmed with pertinent settings.

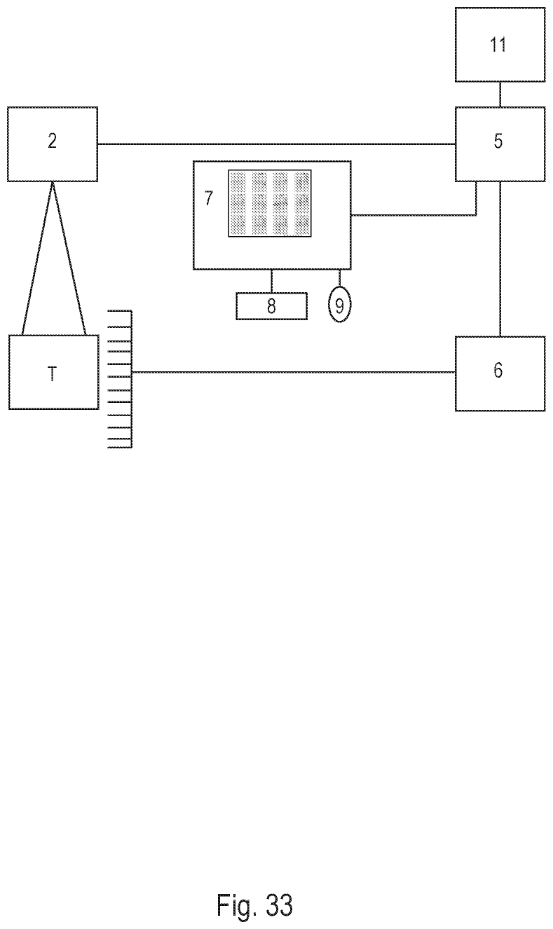

An embodiment of a system (FIG. 33) according to the invention comprises a system for performing a computer implemented method. A computer 5 comprises a processing unit, a with the processing unit functionally coupled memory, a 3D or anatomic model receiver, the model preferably being a torso model and/or a heart model, a location information receiver of at least one heart conduction feature, such as the His Purkinje system or parts, or a model thereof, the base of the papillary muscles, the right free wall moderator band connection, the right apical septal position, the mute apical left septal position and/or the basal left septal position. The computer is preferably coupled with an ECG receiver for receiving (600) ECG recording data, preferably with corresponding 3D torso information, of a currently performed ECG. It is however also envisaged to perform the method at a later time based on previously acquired data, including the 3D model, ECG measurements, for creating an activation map and thereto related renderings or data creations as indicated in this document. The computer comprises a heart activation map determining module relative to the model, such as comprising steps of updating of electrophysiological properties of the model. The system may comprise a three-dimensional camera 2, for detecting ECG electrodes arranged at a torso T, is arranged above the torso T (schematically shown) of a person. The camera is suitable for moving thereof relative to the torso such that from several sides the torso can be recorded for detecting of the ECG electrodes jet in place. Data from the camera are transferred to the computer 5. The computer is connected to a monitor 7, keyboard 8 and mouse 9 for receiving input data from these peripherals from a user and for outputting of image data to the user. The computer is furthermore coupled with an ECG amplifier 6 that in its turn is coupled to ECG electrodes 3 on the torso T. A practical number of electrodes that is supplied is between 4 and 16, preferably substantially 12. A larger number for achieving a higher resolution is envisaged and use thereof dependent on the surroundings in which the installation is applied also usable. The skilled person would be able to determine the number of electrodes as a correct choice based on available equipment.

The computer comprises a set of candidate positions determining module, preferably comprising a target position determining module. The system comprises an ECG device 2 for recording of respective ECG sessions, preferably a standardized ECG session, further preferably a standardized 12 lead ECG session. The computer comprises a location information receiver, preferably as a software module, for receiving (500) location information of at least one cardiac vein, such as by estimation or scanning data. The computer comprises a potential implant areas determining module, for the purpose of determining the areas for implantation of at least one pacing system lead, such as for the purpose of outputting such information relating to the model and the potential implant areas for displaying thereof.

An embodiment of the method or system according to the invention aims to relate the cardiac activation initiated from the His-Purkinje system to the ECG. The human His-Purkinje system distributes the electrical activation to a large part of the endocardial surface of the left and right ventricle. In cardiac activation in humans, the initial activation is typically found on the anterior left septum, with later local breakthroughs in the left and right apical regions as well as the right free wall. The initial activation from a branch of the His-Purkinje system is approximated in preferred embodiments by an endocardial surface being activated almost simultaneously, attributed to the density of the local available Purkinje-myocardial junctions (Purkinje fiber) located on the endocardial surface. Such locations are indicated in FIG. 20 with the indication of Foci in the Cardiac source model or by the endings 3511-3518 of FIG. 35. Thus, the Purkinje initiated ventricular activation is modelled according to preferred embodiments by a combination of multiple breakthroughs in different parts of the left and right ventricular myocardium. In FIG. 35, such breakthroughs are modelled by the endings 3511-3518 as indicated in the 3D model. Each ending is arranged at a node of the 3D model and for determinations of accuracy of the location at such specific node, an ending is moved to another node during such analysis. These modelled endings are subsequently displaced in sensitivity analysis to provide the best location in relation to ECG data, as described in further detail elsewhere in this disclosure.

Activation Sequence Construction

Preferably, a fastest route algorithm is used to compute the activation propagation from the initial sites of activation 2, 3. The fastest route algorithm computes the (virtual) distances between a node and all other nodes on a closed triangulated myocardial surface. The propagation velocity within the myocardium is preferably assumed as anisotropic, with as preferred meaning that velocities perpendicular to the myocardial fiber direction are for example about 2 times slower. To apply this anisotropic propagation velocity the (virtual) distance for transmural connections is made for example two times longer, as the transmural connections are by definition perpendicular to the local fiber direction 2, 4. To mimic the surface activation from the Purkinje system, the local velocity around a node on the ventricular is preferably set to 1.7 m/s with a radius of preferably 15 mm.

Preferably, a model of a 58 old male with average body build and heart orientation is used to estimate the cardiac activation. A preferred source method used to simulate the equivalence of the cardiac activity is the equivalent dipole layer (EDL) 4-8. This model is preferably used to compute the ECG given the activation time at each of the nodes of the ventricular mesh (FIG. 1) of the model, while taking into account the volume conductor effects using the Boundary Element method. The volume conductor model uses the geometries of the thorax, ventricles and ventricular blood cavities. The conductivity of the blood was set to 3 times the value of the rest of the thorax geometry.

Phenomenological Estimation of his-Purkinje System ActivaTion Estimation

To estimate the His-Purkinje activation sequence from a patient's ECG an initial activation sequence is generated to simulate ECG signals. In this approach the Purkinje system network is modeled with the said endings related to nodes of the 3D model of the heart. This initial sequence uses 8 different anatomical locations of potential Purkinje activations sites 3511-3518 (FIG. 20,35). For the LV septum, 3 sites are preferably located near the base, mid septum, and apical septum. Three more locations are preferably selected on the left free wall, associated with papillary muscle locations and thus with potential sites of early His-Purkinje activation. The two positions on the endocardial RV wall represent the entry of the moderator band 9, and the apical right septal region.

For the purpose of performing iterative optimizations or analysis, the timing of each of these 8 regions is preferably set for different waveform patterns. For determining normal activations the initiation times of the left septal wall were preferably set to 0 ms, such as preferably equal to the QRS onset, while the RV and LV activations times are preferably set to 15 ms. For ECGs with a suspected LBBB pattern (QRS duration>=120 ms) the initial timing of the left regions, is preferably delayed to 40 ms for the septal regions and 45 ms for the free wall regions. Similarly for suspected RBBB ECG waveforms (QRS duration>120 ms), the timing of the RV septal region is preferably set to 45 ms, and the RV free wall to 65 ms.

In a subsequent iterative optimization procedure, the timing and position of these eight sites of activation is changed between iterative steps to ultimately obtain the best match between the simulated ECG and measured ECG. In the iterative approach the timing and position of the endings is changed such that the correlation or match between the simulated and measured ECGs is maximized. The total activation duration for each constructed sequence is matched to the QRS duration by adapting the overall used propagation velocity. The used propagation velocity is maintained within the physiological range of the myocardial velocity, i.e. between 0.5-0.85 ms−1 10-14.

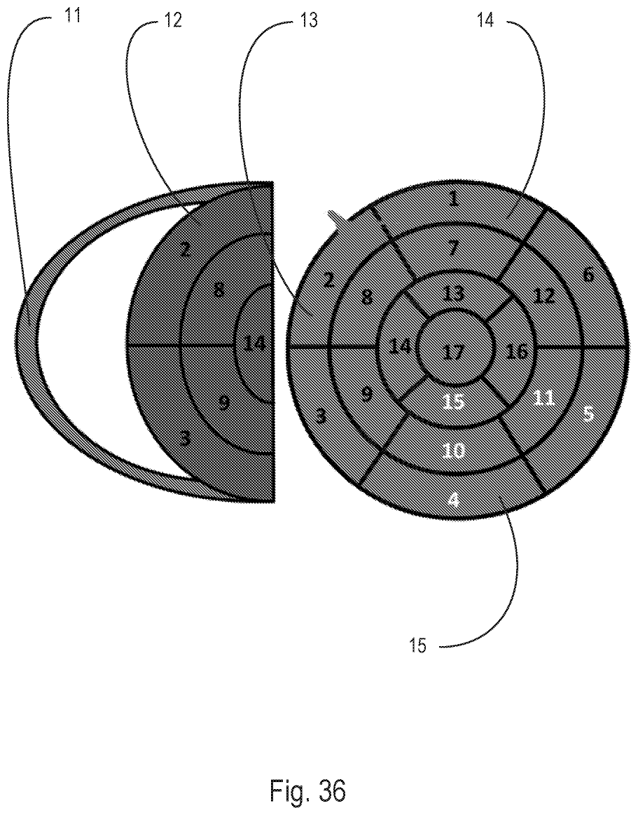

As displayed in FIG. 35, the regional sites of early or initial activation associated with the Purkinje activation. Each ending 3511-3518 identifies a position of the initial estimation of the cardiac activation. The exact position of the His-Purkinje system is unknown, wherein according to these embodiments, the effect of the His-Purkinje system on cardiac activation is determined, such as by simulation FIG. 35. FIG. 36 shows: The 5 regions of the heart: 1)RV free wall, 2) RV septum, 3) LV septum, 4) anterior LV free wall, and 5) posterior LV free wall. The septal and LV free wall segments coincide with a perse known 17 segments model.

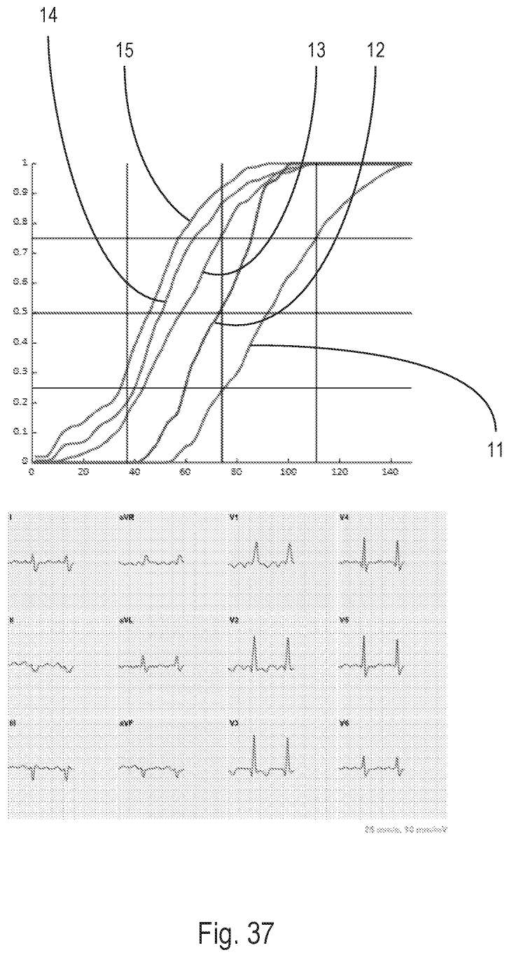

The graphs of FIG. 37-39 provide a representation of the activation map wherein a part of the map representing such region is related to a percentage of mass activated in that part representing such region. This representation of the activation map provides straightforward insights into such region activating outside of ‘normal’ or ‘optimal’ timing, which rendering provides effective insight in asynchrony in a way that was not available in the prior art.

To limit the number of parameters from the complete activation times on the ventricular surface, segment model based regions, indicated as RV free wall 11, RV septum 12, LV septum 13, anterior LV free wall 14, and posterior LV free wall 15 (FIG. 36), are used in preferred embodiments. A finer regional sub division is envisaged. For each of these regions the initial and latest activation time is computed, as well as the percentage mass activated at mid-QRS. This latter parameter, determines the relative amount of the mass represented by the segment being activated before mid-QRS.

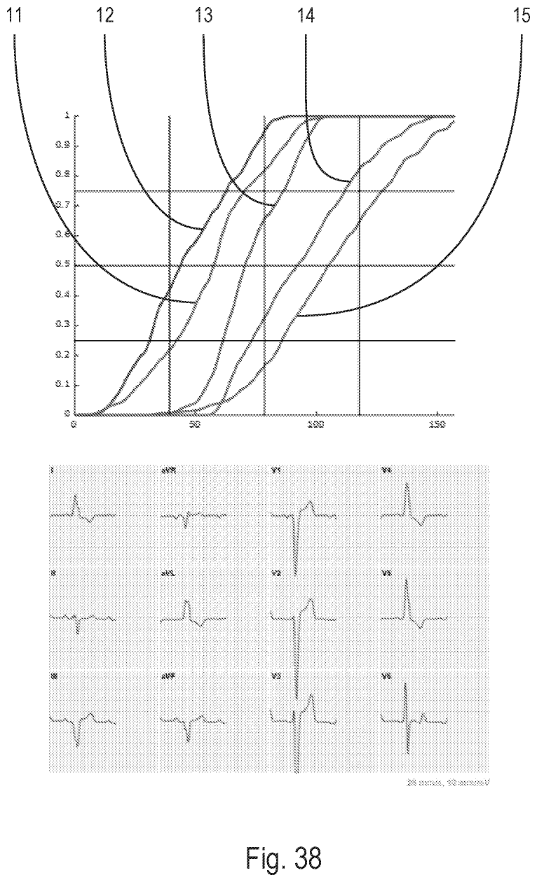

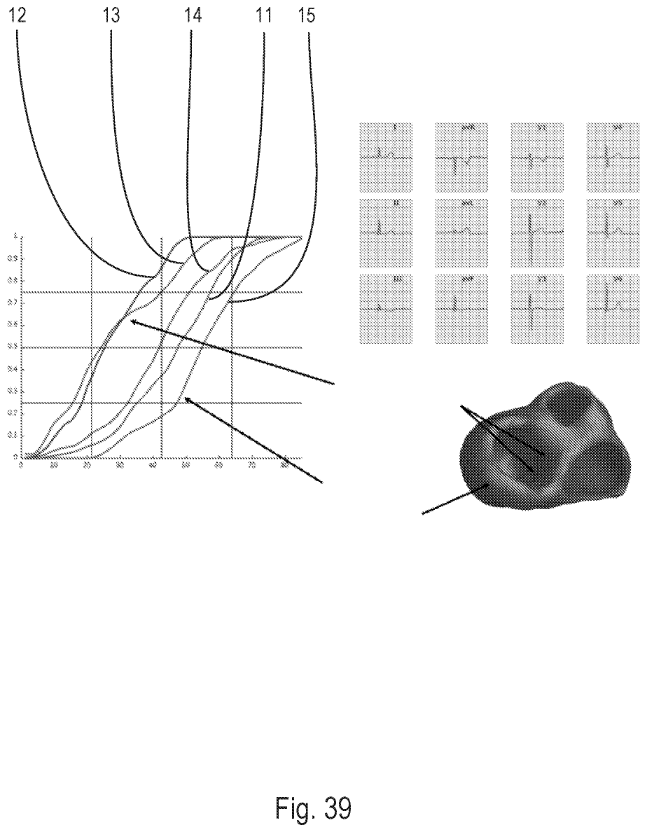

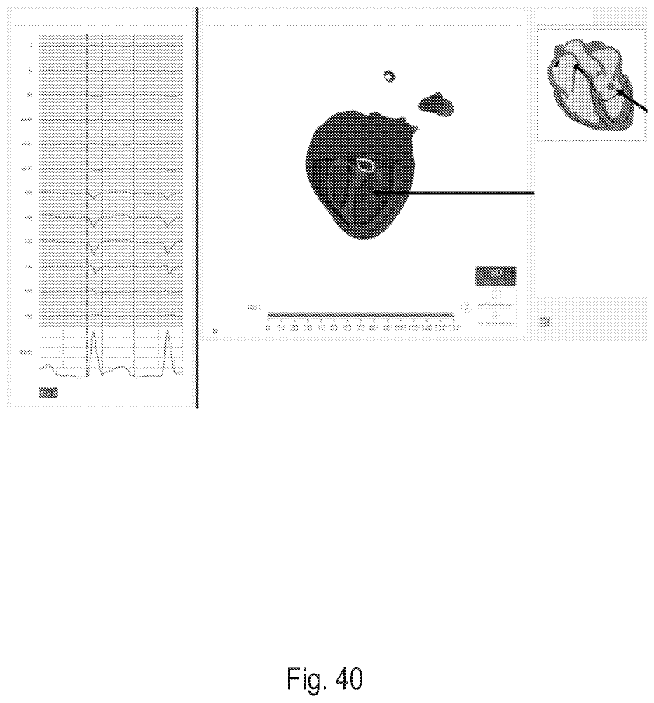

For each of these regions, the percentage mass activated at each activation time in ms is determined for the graphs of FIG. 37 for an example of a typical RBBB and FIG. 38 for a typical LBBB result. FIG. 39 shows a typical average or ‘normal’ result. These relative mass volume graphs show the amount of cardiac tissue being activated over time following an S-curve. The time at which half the segment mass is activated describes this S-curve well. For an RBBB pattern the RV segments are activated late, whereas for the LBBB patterns the LV segments are activated late. The mid QRS time can also be expressed as a relative number expressed in QRS duration.

Such Typical examples of the volume mass activated for the different segments (see FIG. 1): 1)RV free wall, 2) RV septum, 3) LV septum, 4) anterior LV free wall, and 5) posterior LV free wall. The RV wall segments are activated late (after 40% of the QRS duration) for a typical RBBB ECG, whereas the LV segments are late (after 40% of the QRS duration) for an LBBB pattern.

Preferred Parameter Definitions

QRS duration: Time between QRS onset and end

LAT0: Initial activation time for a certain segment (An example of normal activation definition is when the initial activation is less than 25 ms for each segment, delayed when the activation is between 25-40 ms, absent when more than 40 ms).

LATend: Latest activation time per segment, this signifies the area that is severely delayed, and thus does potentially not contrivute optimal to the contraction

% Mass Mid QRS: 50% Mass activated at mid QRS, All segments are preferably at 50% between 30-60 ms.

Velocity: Used propagation velocity (The higher the velocity the more viable the His-Purkinje system is).

Certainty: Correlation between measured and simulated ECG, tell something about the model fit to the individual patient).

VEU: Ventricular electrical uncoupling: average timing of the LV anterior and posterior segments minus the average of the RV free wall segment (This is a published parameter, but now newly computed with endo and epicardial timing

QRS area: The integral of the QRS waveforms in the X,Y,Z signals after a Kors transform of the ECG signals

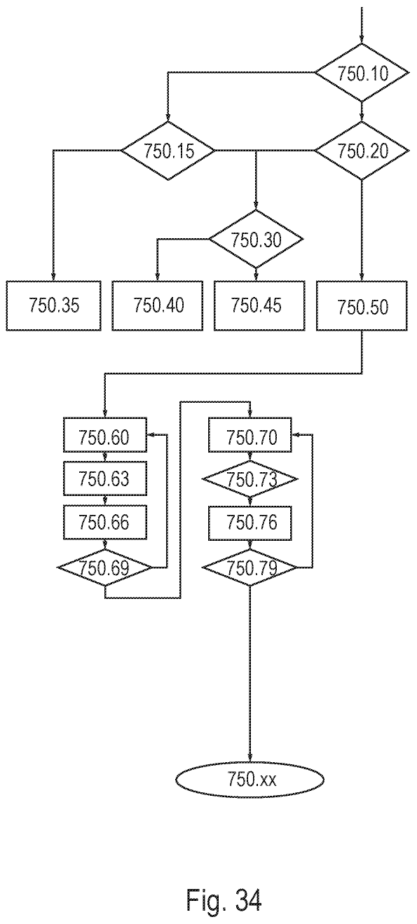

Using the above description, FIG. 34 shows a flowchart relating to the steps comprised by step 750 according to a preferred embodiment. Preferably based on the His-Purkinje foci and the timing matrices, the normal estimated His-Purkinje activation and the scar related activation (the best available for this patient with conduction system) are determined or computed. The His-Purkinje break-through positions and timing are preferably estimated based on features of the ECG/VCG. When the QRS duration is shorter than 100 ms the His-Purkinje system is assumed to be functioning normal.

For steps 750.10-750.50 the timing of the initial foci is preferably set. This is preferably based on whether the direction of the first 15 ms of the QRS VCG is from left to right in step 750.10 and/or whether the QRS duration is normal, such as below 100 ms for steps 750.20, 750.15. For any of these combinations the foci timing is set in steps 750.35-50.

In steps, 750.60-750.79, the timing of every focus is indi-vidually incremented with a time step of for instance 1 ms. Moreover, all directly connected neighbors of the 3D model (connected by a triangle) are tested as well to be a better position of the focus. Depending on results secondary or furthers neighbors may be tested. Based on the adapted timing of the constructed activation sequence the ECG is simulated. When the simulated ECG matches the measured ECG better (for instance the correlation) the timing of this focus is adapted to the better matched value. In the next step a focus is moved to a neighboring node from which the overall timing is computed as well and the resulting simulated ECG is matched to the measured ECG. When the ECG match improved the individual focus is moved. This procedure is repeated until no improvement in timing or focus position is found. With this, an optimal match between the ECG and the model is achieved.

The computation of the activation time on all nodes (i:1:n) from a single focus(k) (ending) is given by:

$$\begin{matrix}{activation}_{k} ( i : 1 : n ) = \begin{matrix}\frac{Distance ( focus ( k ) , i )}{v focus} & d < 15 mm\end{matrix} \\ = \begin{matrix}\frac{Distance ( focus ( k ) , i )}{vmyocard} & d \geq 15 mm\end{matrix}\end{matrix}$$

The computation of the combined focal activation time for all nodes (i) from the given focus (ending) is then:

activationᵢ=MIN(activationₖ(i))ₖ₌₁^{nfoci}

Wherein:

k=number of foci (endings 3511-3518)

i=node

n=# nodes

v=velocity, ex. v focus at 1.5, v myocard at 0.8

A further clause according to the present invention is directed at method, such as implemented on a computer, providing cardiac activation information for rendering thereof, the method comprising steps of:
receiving (100) an 3D or anatomic model and/or processing thereof, such as a torso model and/or a heart model,
receiving (400), such as by estimation or scanning data, location information of at least one heart conduction feature and/or processing thereof, such as the base of the papillary muscles, the right free wall moderator band connection, the right apical septal position, the mute apical left septal position and/or the basal left septal position,
receiving (600) ECG recording data and/or processing thereof, preferably with corresponding 3D torso information, of a currently performed ECG,
determining (700) a heart activation map relative to the model, such as comprising steps of updating of electrophysiological properties of the model. Preferred embodiments of such clause comprise any individual feature is disclosed in this document or a combination thereof.

A main advantage of such heart activation map information is that the activation of distinct parts of the heart may be rendered that are indicative of impairments or inefficiencies in related to the heart, such as a disbalance of the heart activation. It is further preferable that heart conditions underlying information of the activation map may be indicated by being able to observe such rendering. Also representations of the heart activation map or related therewith provide clearly interpretable deviations from a desirable activation map, in absolute sense or related to specific parameters of a heart.

Preferably, a representation is provided by relating segments of the heart to activation timings or times.

Further preferably, such times or timings are related to the mass of the activated segment. Preferably, the activated mass is related to the time or timing a percentage thereof is activated during an activation of the heart.

Preferably, the segment mass or volume in percentage of the segment is rendered in a graph against the activation time, preferably in ms.

The present invention is described in the foregoing on the basis of preferred embodiments. Different aspects of different embodiments are expressly considered disclosed in combination with each other and in all combinations that on the basis of this document, when read by a skilled person of the area of skill, fall within the scope of the invention or are deemed to be read with the disclosure of this document. These preferred embodiments are not limitative for the scope of protection of this document. The rights sought are defined in the appended claims.

1. A method, such as implemented on a computer, providing cardiac pacing therapy guidance, such as providing input for pacing system implant and/or implanted pacing system functioning optimization, for performing heart function optimization, such as comprising pacing system lead positioning guidance, stimulation timing guidance and/or stimulation sequence guidance, the method comprising steps of:
receiving an 3D or anatomic model and/or processing thereof, such as a torso model and/or a heart model,
receiving, such as by estimation or scanning data, location information of at least one heart conduction feature and/or processing thereof, such as the His Purkinje system or parts, or a model thereof, the base of the papillary muscles, the right free wall moderator band connection, the right apical septal position, the mute apical left septal position and/or the basal left septal position,
receiving location information of at least one cardiac vein and/or processing thereof, such as by estimation or scanning data,
receiving ECG recording data and/or processing thereof, preferably with corresponding 3D torso information, of a currently performed ECG,
determining a heart activation map relative to the model, such as comprising steps of updating of electrophysiological properties of the model,
determining potential implant areas for implantation of at least one pacing system lead, such as for the purpose of outputting such information relating to the model and the potential implant areas for displaying thereof.

2. The method according to claim 1, further comprising steps of determining a set of candidate positions for the at least one pacing system lead, preferably determining a target position for the pacing system lead from the set of candidate positions.

3. The method according to claim 1, further comprising steps of processing scanning or recording data or signals pertaining to at least one pacing system lead activation during guidance thereof towards one of the set of candidate positions, preferably the target position, preferably including location information thereof relating to the location of the pacing system lead at the time of the activation.

4. The method according to claim 3, wherein the steps of processing scanning or recording information comprises steps of processing, VCG, data or signals pertaining to the at least one activation of the pacing system lead.

5. The method according to claim 3, wherein the steps of processing scanning or recording information comprises steps of processing inverse ECG data or signals pertaining to the at least one activation of the pacing system lead.

6. The method according to claim 3, wherein the steps of processing scanning or recording information comprises steps of processing pacing spike data or signals, such as by localization, such as by localization of at least one pacing spike from the QRS ECG, pertaining to the at least one activation of the pacing system lead.

7. The method according to claim 1, further comprising steps of including least one intracavitary structure, such as papillary muscles or moderator band, preferably comprising information as to position, size and orientation of such structures.

8. The method according to claim 1, further comprising steps of including data pertaining to dysfunctional or traumatized myocardial tissue, such as areas of scar or ischemic tissue if present in the myocardial tissue.

9. The method according to claim 1, further comprising steps of determining whether the at least one pacing lead is positioned at the target position or within a predetermined deviation threshold thereof.

10. A method, such as implemented on a computer, providing cardiac pacing therapy guidance, such as during pacing system functioning optimization, for performing heart function optimization, such as stimulation timing guidance and/or stimulation sequence guidance, the method comprising steps of:
receiving pacing system, such as CRT, implantation data from a data store and/or processing thereof,
receiving ECG recording data and/or processing thereof, preferably with corresponding 3D torso information, of a currently performed ECG,
determine cardiac pacing therapy value set, such as comprising a synchrony value,
outputting pacing system settings to one of a set of test settings, preferably until all of the settings of the set of test settings have been performed, for the pacing system to be programmed to the respective setting, and
displaying the determined cardiac pacing therapy value set on a display device.

11. The method according to claim 11, further comprising steps of outputting a setting identification identifying the test settings identified as best for applying the settings to the pacing system.

12. A cardiac pacing therapy guidance system, such as comprising a computer, providing cardiac pacing therapy guidance, such as during pacing system implant and/or implanted pacing system functioning optimization, for performing heart function optimization, such as comprising pacing system lead positioning guidance, stimulation timing guidance and/or stimulation sequence guidance, the system comprising:
a processing unit,
a with the processing unit functionally coupled memory,
a 3D or anatomic model receiver, the model preferably being a torso model and/or a heart model,
a location information receiver of at least one heart conduction feature, such as the His Purkinje system or parts, or a model thereof, the base of the papillary muscles, the right free wall moderator band connection, the right apical septal position, the mute apical left septal position and/or the basal left septal position,
an ECG receiver for receiving ECG recording data, preferably with corresponding 3D torso information, of a currently performed ECG,
a heart activation map determining module relative to the model, such as comprising steps of updating of electrophysiological properties of the model.

13. The system according to claim 12, further comprising a set of candidate positions determining module, preferably comprising a target position determining module.

14. The system according to claim 12, further comprising an ECG device for recording of respective ECG sessions, preferably a standardized ECG session, further preferably a standardized 12 lead ECG session.

15. The system according to claim 14, further comprising a 3D imaging module for recording of 3D torso information, of the torso to which a respective EGG is performed.

16. The system according to claim 12, further comprising an output means for outputting of a user interface, preferably a graphical user interface for displaying any information processed.

17. The system according to claim 12, further comprising a location information receiver for receiving location information of at least one cardiac vein, such as by estimation or scanning data.

18. The system according to claim 12, further comprising a potential implant areas determining module, for the purpose of determining the areas for implantation of at least one pacing system lead, such as for the purpose of outputting such information relating to the model and the potential implant areas for displaying thereof.

19. The system according to claim 12, further comprising input means for inputting user operations, such as on the graphical user interface.
